(19) United States
(12) Patent Application Publication
(10) Pub. No.: US 2005/0239892 A1
(43) Pub. Date: Oct. 27, 2005

(54) Therapeutic avenathramide compounds

(75) Inventor: Mohsen Meydani, Newton, MA (US)

Correspondence Address: NUTTER MCCLENNEN & FISH LLP, WORLD TRADE CENTER WEST, 155 SEAPORT BOULEVARD, BOSTON, MA, 02210-2604, US

(73) Assignee: TRUSTEES OF TUFTS COLLEGE, Medford, MA (US)

(21) Appl. No.: 10/995,722

(22) Filed: Nov. 22, 2004

Related U.S. Application Data
(60) Provisional application No. 60/524,327, filed on Nov. 21, 2003.
(60) Provisional application No. 60/625,484, filed on Nov. 5, 2004.

(51) Int. Cl. A61K 31/195; A61K 35/78

(52) U.S. Cl. 514/563; 424/750

(57) ABSTRACT

Methods and compositions are disclosed for reducing pro-inflammatory molecules, adhesion molecules, and vascular smooth muscle cell proliferation, and for increasing NO production. The present invention describes the use of phenolic compositions, purified from oats or synthetically produced, to decrease the effective amount of pro-inflammatory molecules and/or cell adhesion molecules. Alternatively, an alcoholic extract or concentrate from oats can be used. The methods of the present invention can be used as a treatment or prophylaxis of a wide variety of disorders associated with inflammatory states and/or with a lack of or need for nitric oxide (NO), such as inflammatory conditions, pain, free radical associated disorders, cardiovascular diseases, autoimmune disorders, pathological platelet aggregation, pathological vasoconstriction, vascular effects of diabetes, stroke, atherosclerosis, hypertension, abnormal vasospasm, and restenosis after angioplasty.

### RELATED APPLICATIONS

This application claims priority from U.S. Provisional Application Ser. No. 60/524,327, filed Nov. 21, 2003, entitled “Oat-Derived Therapeutic Compositions” and U.S. Provisional Application Ser. No. 60/625,484, filed Nov. 5, 2004, entitled “Modulation of Nitric Oxide Production And Cell Proliferation Using Oat-Derived Phenolic Compounds,” each of which are hereby incorporated by reference in their entirety.

### GOVERNMENT SUPPORT

This invention was made with government support under 58-1950-9-001 awarded by the United States Department of Agriculture. The government has certain rights in the invention.

### FIELD OF THE INVENTION

The present invention concerns phenolic compositions and extracts derived from oats and methods of using such compositions as therapeutic agents.

### BACKGROUND OF THE INVENTION

Cardiovascular disease (CVD) kills more Americans than any other major cause of death, according to the American Heart Association's Heart Disease and Stroke Statistics (2004). Vessel wall inflammation is a major factor in the development of atherosclerosis, atheroma instability and plaque disruption, which, when followed by local thrombosis, underlies the clinical presentation of acute cardiovascular disease.

Arterial endothelium can change in response to both external and internal stimuli. Elevated and modified LDL, cigarette smoking, hypertension, diabetes mellitus, genetic alterations, increase of plasma homocysteine, and infectious microorganisms, such as herpes virus, are as possible causes of endothelial dysfunction. In atherosclerosis and other diseases, dysfunctional vascular endothelium leads to leukocyte recruitment. In altered arterial endothelium there is increased monocyte adhesion as well as impaired nitric oxide production and vascular relaxation (Cybulsky et al. Science 1991; 251:788-791). Adherence of monocytes to the endothelial surface is facilitated by the expression of the adhesion molecules vascular cell adhesion molecule-1 (VCAM 1) and intercellular adhesion molecule-1 (ICAM-1).

During the initial phase of vessel wall inflammation, the patient usually does not present with clinical symptoms and, thus, the atherosclerosis preclinical window is fairly long. Leukocyte adhesion to the endothelium also occurs early in the pathogenesis of a wide range of inflammatory conditions, including not only atherosclerosis and other cardiovascular diseases, but also autoimmune diseases, as well as bacterial and viral infections. Leukocyte recruitment begins when the endothelial cells produce adhesion molecules (i.e., vascular cell adhesion molecule-1 (VCAM-1), intracellular adhesion molecule-1 (ICAM-1), and E-selectin) that interact with specific leukocytes (i.e., monocytes, lymphocytes, or neutrophils). The extracellular matrix characteristic of the atherosclerotic plaque is propagated by the production of pro-inflammatory cytokines (i.e., IL-6 and IL-8) and chemoattractants that are produced and released following conversion of leukocytes to foamy macrophages.

Nitric oxide (NO) is a regulatory molecule that plays a vital role in the normal physiology of the cardiovascular, intestinal, central nervous, and immune systems. The role of NO, as either a beneficial physiological mediator, or as pathological cytotoxic radical, is largely determined by the level and extent of synthesis. The synthesis of NO from the semi-essential amino acid L-arginine is catalyzed by three different enzyme isoforms, endothelial nitric oxide synthase NOS (eNOS), neuronal NOS(NNOS) and inducible NOS (iNOS). Endothelial NOS (eNOS) and neuronal NOS(NNOS) are constitutively expressed, calcium dependent enzymes, while inducible NOS (iNOS) is not always expressed under physiological conditions.

Increasing the levels of NO in the vasculature is beneficial in many pathologic conditions, such as atherosclerosis, hypertension, diabetes, and stroke. In the vasculature, endothelial derived NO has several actions among which are the inhibition of platelet aggregation, adhesion of inflammatory cells, and the proliferation of smooth muscle cells. Endothelial derived NO is an important regulator of vascular tone.

The mechanism for the regulation of vascular tone by NO is initiated by stimuli, such as acetylcholine, bradykinin, shear stress, etc., on the endothelial, lining cells. The endothelial cells respond by producing NO from L-arginine by eNOS. The NO produced leaves the endothelial cells and stimulates the activity guanylate cyclase in the adjoining smooth muscle cells. Activation of guanylate cyclase increases the level of cGMP and causes the smooth cell to relax, thus dilating the vessel and increasing the blood flow. (Moncada et al., New Eng. J. Med., 329, pp. 2002-2012 (1993)).

Reduced endothelial NO generation may lead to impaired vasodilatation, abnormal vasospasm, increased platelet aggregation, and increased adhesion and infiltration of inflammatory cells. Impairment of endothelial NO and endothelial function are associated with the risk factors for coronary artery disease including smoking, hypercholesterolemia, homocycteinuria, and diabetes. Alteration of NO modulated activities in the coronary arteries may contribute to acute coronary syndrome leading to myocardial infarction. Impairment of the endothelial NO system and its resulting vasoconstriction have been implicated in exacerbating the damage to neurons in cerebral ischemic events, such as, stroke. Additionally, recent studies indicate that endothelial NO mediates the vascular sensitivity to insulin, thus enhanced NO production may be useful in treating the vascular effects of diabetes.

Current medical treatments of cardiovascular disease are not satisfactory since a lot of the damage to the artery walls has already been done by time medication is given. Anticoagulant drugs have been used to try to minimize secondary clotting and embolus formation, but have little or no effect on the progress of the disease. Vasodilator drugs are used to provide symptom relief, but are of no curative value. Current therapy to enhance NO levels in the vasculature has been either to administer high doses of L-arginine, or compounds such as nitroglycerine or sodium nitroprusside, which metabolically release NO. These therapies suffer from undesirable side-effects and their inability to maintain a sustained release of NO, due to their rapid clearance from the body. Surgical treatments are also associated with many health risks. For example, balloon angioplasty, which can be used to open up narrowed vessels and increase blood flow, can lead to permanent damage to a valve or blood vessel, as well as, a risk of restenosis, infection or thrombosis.

There exists a need for better methods and compositions for the treatment of cardiovascular disease and immune disorders. In particular, new compositions capable of modulating inflammatory and/or atherogenic responses would satisfy a long-felt therapeutic need. There is also a need in the art for better methods and compositions for the treatment of disorders associated with abnormal NO production, such as atherosclerosis, diabetes, stroke, and hypertension. In particular, new compositions capable of modulating the nitric oxide pathway would satisfy a long-felt therapeutic need.

### SUMMARY OF THE INVENTION

Methods and compositions are disclosed for reducing pro-inflammatory molecules, adhesion molecules, and vascular smooth muscle cell proliferation, and for increasing NO production. The present invention describes the use of phenolic compositions, purified from oats or synthetically produced, to decrease the effective amount of pro-inflammatory molecules and/or cell adhesion molecules. Alternatively, an alcoholic extract or concentrate from oats can be used. The methods of the present invention can be used as a treatment or prophylaxis of a wide variety of disorders associated with inflammatory states and/or with a lack of or need for nitric oxide (NO), such as inflammatory conditions, pain, free radical associated disorders, cardiovascular diseases, autoimmune disorders, pathological platelet aggregation, pathological vasoconstriction, vascular effects of diabetes, stroke, atherosclerosis, hypertension, abnormal vasospasm, and restenosis after angioplasty.

In one aspect of the present invention, human aortic smooth muscle cell (HASMC) proliferation can be reduced and NO production can be increased using an alcoholic oat extract and/or phenolic compounds. In another aspect, pro-inflammatory cytokines and cell adhesion molecules can be inhibited using an alcoholic oat extract and/or phenolic compounds. Non-limiting examples of pro-inflammatory molecules include IL-6, IL-8, and MCP-1. Non-limiting examples of cell adhesion molecules include ICAM-1, VCAM-1, and E-selectin.

The invention makes use of an extract from oats and/or the synthetic purification of these phenolic compounds. The phenolic compounds can have the core structure shown below: 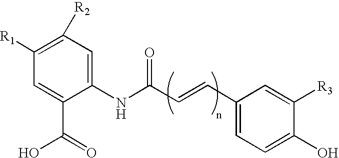
where n is less than or equal to six, and R₁, R₂, and R₃ depict the various side chains which can include, but are not limited to, a hydroxide, an aliphatic group, an aromatic group, an acyl group, an alkoxy group, an alkylene group, an alkenylene group, an alkynylene group, a hydroxycarbonylalkyl group, an anhydride, an amide, an amine, and a heterocyclic aromatic group. In one embodiment, n is less than three and the side chains are selected from the group consisting of H, OH, or OCH₃.

In a preferred embodiment, the phenolic compounds are avenanthramides. The avenanthramides may be purified from grain. More than 40 distinct avenanthramides have been isolated from oat grains (Collins. J. Agric. Food Chem. 37: 60-66 (1989)). In another aspect of the invention, the avenanthramides are produced synthetically. Methods of synthesis are known in the art as illustrated in U.S. Pat. Nos. 6,096,770 and 6,127,392 as well as Japanese Patent No. J60019-754-A and Hungarian Patent HU 200 996 B, which are herein incorporated by reference. One preferred compound comprises Avenanthramide C (Av-C).

In another embodiment, the present invention can be used as a nutraceutical formulation, additive, or supplement. In one embodiment, the present invention could be used to produce supplements containing extracted and purified phenolic compounds. In a preferred embodiment, the food supplement would comprise purified avenanthramides. In another embodiment, the oat alcoholic extract and/or avenanthramides could be incorporated into nutritional supplements that may be added to one's diet for beneficial health effects.

The proliferation of intimal vascular smooth muscle cells (SMC) and impaired NO production are both crucial pathophysiological processes in the initiation and development of atherosclerosis. The methods of the invention can be used to increase NO production. The invention discloses that synthetic avenanthramides can increase NO production in both human aortic smooth muscle cells (HSMC) and human aortic endothelial cells (HAEC). Avenanthramides can also increase expression of endothelial nitric oxide synthase (eNOS) by SMC and endothelial cells. In addition, the invention provides methods of inhibiting proliferation of human aortic smooth muscle cell (HSMC).

The methods of the invention can be used in the reduction, treatment or prophylaxis of conditions caused by modified production of nitric oxide. Non-limiting examples of such conditions include cardiovascular diseases, pathological platelet aggregation, pathological vasoconstriction, vascular effects of diabetes, stroke, atherosclerosis, hypertension, abnormal vasospasm, and restenosis after angioplasty. In one embodiment, a pharmaceutical composition could be made comprising a phenolic composition, or a pharmaceutically acceptable salt thereof, and a pharmaceutically acceptable carrier or diluent.

In one embodiment, the methods of the present invention are useful in modulating atherosclerosis and restenosis after angioplasty. Compounds of the present invention can be incorporated onto a variety of stents to prevent proliferation of SMC and prevent restenosis following application of stents. In yet another embodiment, methods of the present invention can be used to reduce blood pressure through increasing NO.

The methods of the invention can also be used to decrease cell expression of adhesion molecules, production of chemokines and pro-inflammatory of cytokines. Endothelial cells do not normally express elevated levels of adhesion molecules and thus, do not normally support excessive attachment to leukocytes. However, such an interaction is stimulated by exposure to a number of stimuli, including LDL, oxidized LDL, bacterial lipopolysaccharide, and inflammatory cytokines such as IL-1β, which induces phenotypic changes. The cytokine- and/or chemokine-stimulated adhesion of monocytes to endothelium of bovine, porcine, and human origin has been routinely used as a model to investigate interactions between leukocytes and the endothelium. Pretreatment of HAEC with oat extracts dose-dependently reduced IL-1β stimulated HAEC expression of adhesion molecules, productions of chemokines and pro-inflammatory of cytokines and their adherence to U937 cells.

The methods of the invention can be used in the reduction, treatment or prophylaxis of conditions caused by modified production and/or secretion of pro-inflammatory molecules or cell adhesion molecules. Non-limiting examples of such conditions include inflammatory conditions, free radical associated disorders, pain, autoimmune diseases, cardiovascular diseases, and atherosclerosis. In one embodiment, a pharmaceutical composition could be made comprising a phenolic composition, or a pharmaceutically acceptable salt thereof, and a pharmaceutically acceptable carrier or diluent. In another embodiment, the phenolic composition could be used as a topical lotion to treat inflammatory conditions.

The methods of the present invention represent a significant improvement over readily available treatment of cardiovascular disease and inflammation. Since leukocyte adhesion to the endothelium occurs early in the pathogenesis of atherosclerosis and inflammation, the methods of the present invention may be used, for example, to prevent new lesions or atherosclerotic plaques from forming, as well as enabling previously developed lesions to regress. Therefore, the present invention has the possibility to cure the disease or prevent its occurrence, which is a vast improvement over the currently available treatments that simply attempt to slow disease progression.

### BRIEF DESCRIPTION OF THE DRAWINGS

FIG. 1 is a bar graph showing oat extracts and DMSO cytotoxicity on confluent human aortic endothelial cells (HAEC);

FIG. 2 is a bar graph comparing the effects of 24 h incubation with varying concentrations of oat extracts on monocyte-endothelial cell adhesion;

FIG. 3A is a bar graph comparing the effects of 24 h incubation with varying concentrations of oat extracts on HAEC expression of the adhesion molecule intracellular adhesion molecule-1 (ICAM-1);

FIG. 3B is a bar graph comparing the effects of 24 h incubation with varying concentrations of oat extracts on HAEC expression of the adhesion molecule vascular cell adhesion molecule-1 (VCAM-1);

FIG. 3C is a bar graph comparing the effects of 24 h incubation with varying concentrations of oat extracts on HAEC expression of the adhesion molecule endothelial leukocyte adhesion molecule-1 (E-selectin);

FIG. 4A is a bar graph comparing the effects of 24 h incubation with varying concentrations of oat extracts on constitutive HAEC expression of interleukin-8 (IL-8);

FIG. 4B is a bar graph comparing the effects of 24 h incubation with varying concentrations of oat extracts on IL-1β stimulated HAEC expression of IL-8;

FIG. 5 is a bar graph comparing the effects of 24 h incubation with varying concentrations of oat extracts on IL-li stimulated HAEC expression of IL-6;

FIG. 6A is a bar graph comparing the effects of 24 h incubation with varying concentrations of oat extracts on constitutive HAEC expression of monocyte chemoattractant protein-1 (MCP-1);

FIG. 6B is a bar graph comparing the effects of 24 h incubation with varying concentrations of oat extracts on IL-1β stimulated HAEC expression of MCP-1;

FIG. 7 is a bar graph showing that treatment of human aortic smooth muscle cells (HSMC) with avenanthramides inhibits FBS-induced DNA synthesis;

FIG. 8A is a graph showing that avenanthramides inhibit proliferation of human aortic smooth muscle cells (HSMC);

FIG. 8B is a graph showing that avenanthramides inhibit proliferation of A10 cells (rat embryonic aortic smooth muscle cells);

FIG. 9A is a bar graph showing that avenanthramides dose-dependently increased NO production of human aortic smooth muscle cells (HSMC);

FIG. 9B is a bar graph showing that avenanthramides dose-dependently increased NO production of human aortic endothelial cells (HAEC);

FIG. 10A is a bar graph showing that avenanthramides dose-dependently increased eNOS mRNS expression of human aortic smooth muscle cells (HSMC) as detected by real time PCR; and

FIG. 10B is a bar graph showing that avenanthramides dose-dependently increased eNOS mRNA expression of human aortic endothelial cells (HAEC) as detected by real time PCR.

### DETAILED DESCRIPTION OF THE INVENTION

The present invention is directed to utilizing avenanthramides and/or oat extracts to increase NO production, to inhibit vascular smooth muscle cell proliferation and to modulate immune responses. The methods of the invention can be used to decrease production, secretion, and release of immune molecules such as pro-inflammatory molecules and cell adhesion molecules. The present invention can be used to treat, reduce, and/or prevent diseases resulting from modified immune responses including, but not limited to, inflammatory disorders, pain, autoimmune diseases, cardiovascular diseases, free radical associated disorder, and atherosclerosis.

So that the invention is more clearly understood, the following terms are defined:

The term “avenanthramide” as used herein refers to nitrogen containing phenolic compounds. The substituted N-cinnamolylanthranilate alkaloids occur naturally and can be purified from oat groats or hulls where they appear to be most concentrated in the peripheral regions. The avenanthramides comprise conjugated forms of aminophenolic acids, anthranilic, 5-hydroxyanthranilic, 4-hydroxanthranilic. Various hydroxy/methoxy substituted cinnamic or phenylpentadienoic acids attached via linkage to the amide of the aminophenolic moiety comprise the conjugated forms. More than 50 distinct avenanthramides have been isolated from oat grains.

The term “phenolic compounds” as used herein refers to a member of a class of organic molecules which have an aromatic ring with one or more hydroxyl substituents. These compounds comprise a wide spectrum of plant substances and frequently occur attached to sugars. Phenolic compounds, are powerful antioxidants that are found in potatoes, tomatoes, peppers, parsley, squash, yams, celery, carrots, cabbage, soybeans, flaxseed, whole grains, fruits, including citrus, some nuts and garlic. More than 200 phenolic compounds have been identified. Flavonoids, C15 compounds composed of two phenolic rings connected by a three-carbon unit, are the largest group of phenolic compounds.

Several phenolic compounds, such as avenanthramides, caffeic acid, ferulic acid, vanillic acid, sinapic acid, p-coumaric acid, and p-hydroxybenzoic acid flavonoids, have been identified in oats (Peterson, D. J. Cereal Sci. 33: 115-129 (2001)). From analytical and structural chemistry standpoints, these can be roughly divided into low molecular weights, readily soluble “free phenolics” (such as tocols, flavonoids, hydroxycinnamates, etc.), and “bound phenolics,” those covalently linked to complex high molecular weight, insoluble cell components (such as lignin, cell wall polysaccharides, structural protein, etc.). The “free phenolics” appear to represent readily absorbed sources of antioxidants in the human diet, while insoluble “bound phenolics,” requiring further metabolism before absorption from the gastrointestinal tract, present different challenges in attempts to evaluate the long-term efficacy of these compounds. Unlike other cereals however, oats contain a unique source of low molecular weight soluble phenolics, the avenanthramides, not present in other cereal grains, which exhibit potent antioxidant properties. These antioxidants constitute by far the major phenolic antioxidants present in the kernel. They occur in relatively high concentrations in the outer regions of the oat kernel, (e.g. bran and sub-aleurone layers) (Dimberg, L. H. et al. Cereal Chem. 70: 637-641 (1992)), although they are not restricted to these tissues.

A group of phenolic compounds can be described by the core structure shown below, 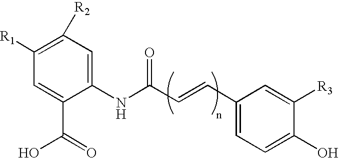
where n is less than or equal to six and R₁, R₂, and R₃ can be, but are not limited to a hydroxide, an aliphatic group, an aromatic group, an acyl group, an alkoxy group, an alkylene group, an alkenylene group, an alkynylene group, a hydroxycarbonylalkyl group, an anhydride, an amide, an amine, and a heterocyclic aromatic group, In a preferred embodiment, n is one or two and R₁, R₂, and R₃ are selected from the group consisting of H, OH, or OCH₃.

The above structure is intended to include the various isomers that exist. Collins et al. (Collins et al. J. Chromatogr. 445: 363-370 (1988)) showed that phenolic compounds, such as avenanthramides, easily undergo Z-E rearrangement and that the E isomers appear to be more easily isolated. Isomers often can have very different activity. For example, Kakegawa and coworkers showed that the Z form of N-(3′,4′-dimethoxycin-namoyl)anthranilic acid has over 10 times the antiallergic activity as the E form.

The term “extract”, as used herein, is meant to encompass a compound or mixture of compounds that are obtained from oats. The extract can be obtained by extraction or distillation of any oat species, fresh or dried, or parts thereof, and is intended to incorporate any isomers that form or can form. Altering the composition of the solvent can change the extract composition, thus enhancing or reducing the biological activity. Work by Collins and co-workers resulting in U.S. Pat. No. 5,169,660 was able to show for the first time that phenolic compounds, known as avenanthramides, occur naturally and can be extracted from oat grain.

The terms “purified” and “substantially purified,” as used interchangeably herein, refer to a compound that is at least 60%, by weight, free from proteins and naturally-occurring organic molecules with which it is naturally associated. Preferably the preparation is at least 75%, more preferably 90%, and most preferably at least 99%, by weight, chemical compound, e.g., Avenanthramide C. A purified compound may be obtained, for example, by high pressure liquid chromatograph, thin layer chromatography, or by synthesizing it.

The term “aliphatic” as used herein refers to open-chain (non-cyclic) hydrocarbons. Aliphatic is especially used in reference to open-chain (non-cyclic) hydrocarbons. The term also refers to open-chain hydrocarbon sub-units of larger organic molecules. The aliphatic group may be further substituted by additional aliphatic or aromatic groups. Non-limiting examples of aliphatic groups consist of alkyl, alkenyl, alkynyl, cycloalkyl, cycloalkenyl, heterocyclyl, heterocyclenyl, aralkenyl, aralkyloxyalkyl, aralkyloxycarbonylalkyl, aralkyl, aralkynyl, aralkyloxyalkenyl, heteroaralkenyl, heteroaralkyl, heteroaralkyloxyalkenyl, heteroaralkyloxyalkyl, heteroaralkynyl, fused arylcycloalkyl, fused heteroarylcycloalkyl, fused arylcycloalkenyl, fused heteroarylcycloalkenyl, fused arylheterocyclyl, fused heteroarylheterocyclyl, fused arylheterocyclenyl, fused heteroarylheterocyclenyl, and the like. “Aliphatic”, as used herein, also encompasses the residual, non-carboxyl portion of natural and unnatural amino acids as defined herein.

The term “aromatic” as used herein refers to both aryl and heteroaryl rings. The aryl or heteroaryl ring may be further substituted by additional aliphatic or aromatic radicals. Representative aromatic groups include aryl, fused cycloalkenylaryl, fused cycloalkylaryl, fused heterocyclylaryl, fused heterocyclenylaryl, heteroaryl, fused cycloalkylheteroaryl, fused cycloalkenylheteroaryl, fused heterocyclenylheteroaryl, fused heterocyclylheteroaryl, and the like.

The term “acyl” as used herein refers to an H—CO— or alkyl-CO- group. Preferred acyl groups contain a lower alkyl, formyl, acetyl, propanoyl, 2-methylpropanoyl, butanoyl or palmitoyl.

The term “acylamino” as used herein refers to an acyl-NH-group.

The term “alkenyl” as used herein refers to a straight or branched aliphatic hydrocarbon group of 2 to about 15 carbon atoms which contains at least one carbon-carbon double bond. Preferred alkenyl groups have 2 to about 12 carbon atoms; more preferred alkenyl groups have 2 to about 4 carbon atoms. The alkenyl group may be substituted with one or more alkyl group substituents as defined herein. Representative alkenyl groups include ethenyl, propenyl, n-butenyl, i-butenyl, 3-methylbut-2-enyl, n-pentenyl, heptenyl, octenyl, cyclohexylbutenyl and decenyl.

The term “alkoxy” as used herein refers to an alkyl-O-group wherein the alkyl group is as defined herein. Representative alkoxy groups include methoxy, ethoxy, n-propoxy, i-propoxy, n-butoxy, heptoxy, and the like.

The term “alkyl” as used herein refers to an aliphatic hydrocarbon group, which may be straight or branched-chain, having about 1 to about 20 carbon atoms in the chain. Preferred alkyl groups have 1 to about 12 carbon atoms in the chain. Branched means that one or more lower alkyl groups such as methyl, ethyl or propyl are attached to a linear alkyl chain. “Lower alkyl” means 1 to about 4 carbon atoms in the chain, which may be straight or branched. The alkyl may be substituted with one or more “alkyl group substituents” which may be the same or different, and include halo, cycloalkyl, hydroxy, alkoxy, amino, carbamoyl, acylamino, aroylamino, carboxy, alkoxycarbonyl, aralkyloxycarbonyl, or heteroaralkyloxycarbonyl. Representative alkyl groups include methyl, trifluoromethyl, cyclopropylmethyl, cyclopentylmethyl, ethyl, n-propyl, i-propyl, n-butyl, 1-butyl, n-pentyl, 3-pentyl, methoxyethyl, carboxymethyl, methoxycarbonylethyl, benzyloxycarbonylmethyl, and pyridylmethyloxycarbonylmethyl.

The term “alkylene” as used herein refers to a straight or branched bivalent hydrocarbon chain of 1 to about 6 carbon atoms. The alkylene may be substituted with one or more “alkylene group substituents” which may be the same or different, and include halo, cycloalkyl, hydroxy, alkoxy, carbamoyl, carboxy, cyano, aryl, heteroaryl or oxo. Preferred alkylene groups are the lower alkylene groups having 1 to about 4 carbon atoms. Representative alkylene groups include methylene, ethylene, and the like.

The term “alkenylene” as used herein refers to a straight or branched bivalent hydrocarbon chain containing at least one carbon-carbon double bond. The alkenylene may be substituted with one or more “alkylene group substituents” as defined herein. Representative alkenylene include —CH═CH—, —CH₂ CH═CH—, —C(CH₃)═CH—, —CH₂CH═CHCH₂—, and the like.

The term “alkynylene” as used herein refers to a straight or branched bivalent hydrocarbon chain containing at least one carbon-carbon triple bond. The alkynylene is optionally substituted with one or more “alkylene group substituents” as defined herein. Representative alkynylene include —C≡C—, —C≡C—CH₂—, —C≡C—CH(CH₃)—, and the like.

The term “alkynyl” used herein refers to a straight or branched aliphatic hydrocarbon group of 2 to about 15 carbon atoms which contains at least one carbon-carbon triple bond. Preferred alkynyl groups have 2 to about 12 carbon atoms. More preferred alkynyl groups contain 2 to about 4 carbon atoms. “Lower alkynyl” means alkynyl of 2 to about 4 carbon atoms. The alkynyl group may be substituted by one or more alkyl group substituents as defined herein. Representative alkynyl groups include ethynyl, propynyl, n-butynyl, 2-butynyl, 3-methylbutynyl, n-pentynyl, heptynyl, octynyl, decynyl, and the like.

The term “amino” used herein refers to a group of formula Z¹ Z² N- wherein Z¹ and Z² are independently hydrogen; acyl; or alkyl, or Z¹ and Z² taken together with the N through which Z¹ and Z² are linked to form a 4 to 7 membered azaheterocyclyl. Representative amino groups include amino (H₂N—), methylamino, dimethylamino, diethylamino, and the like.

The term “aminoalkyl” used herein refers to an amino-alkylene-group wherein amino and alkylene are defined herein. Representative aminoalkyl groups include aminomethyl, aminoethyl, dimethylaminomethyl, and the like.

The term “aralkyl” used herein refers to an aryl-alkylene-group wherein aryl and alkylene are as defined herein. Preferred aralkyls contain a lower alkyl moiety. Representative aralkyl groups include benzyl, 2-phenethyl, naphthlenemethyl, and the like.

The term “aroyl” used herein refers to an aryl-CO— group wherein aryl is defined herein. Representative aroyl include benzoyl, naphth-1-oyl and naphth-2-oyl.

The term “cycloalkyl” used herein refers to a non-aromatic mono- or multicyclic ring system of about 3 to about 10 carbon atoms, preferably of about 5 to about 10 carbon atoms. Preferred cycloalkyl rings contain about 5 to about 6 ring atoms. The cycloalkyl can be substituted with one or more “ring system substituents” which may be the same or different, Representative multicyclic cycloalkyl include 1-decalin, norbornyl, adamantyl, and the like.

There term “cycloalkenyl” used herein refers to a non-aromatic mono- or multicyclic ring system of about 3 to about 10 carbon atoms, preferably of about 5 to about 10 carbon atoms which contains at least one carbon-carbon double bond. Preferred cycloalkylene rings contain about 5 to about 6 ring atoms. The cycloalkenyl may be substituted with one or more “ring system substituents” which may be the same or different, and are as defined herein. Representative monocyclic cycloalkenyl include cyclopentenyl, cyclohexenyl, cycloheptenyl, and the like. A representative multicyclic cycloalkenyl is norbornylenyl.

There term “aryl” used herein refers to an aromatic monocyclic or multicyclic ring system of 6 to about 14 carbon atoms, preferably of 6 to about 10 carbon atoms. The aryl may be substituted with one or more “ring system substituents” which may be the same or different, and are as defined herein. Representative aryl groups include phenyl and naphthyl.

The term “heteroaryl” used herein refers to an aromatic monocyclic or multicyclic ring system of about 5 to about 14 ring atoms, preferably about 5 to about 10 ring atoms, in which one or more of the atoms in the ring system is/are element(s) other than carbon, for example nitrogen, oxygen or sulfur. Preferred heteroaryls contain about 5 to about 6 ring atoms. The “heteroaryl” is optionally substituted by one or more “ring system substituents” which may be the same or different, and are as defined herein. The prefix aza, oxa or thia before heteroaryl means that at least a nitrogen, oxygen or sulfur atom respectively is present as a ring atom. A nitrogen atom of a heteroaryl is optionally oxidized to the corresponding N-oxide. Representative heteroaryls include pyrazinyl, furanyl, thienyl, pyridyl, pyrimidinyl, isoxazolyl, isothiazolyl, oxazolyl, thiazolyl, pyrazolyl, furazanyl, pyrrolyl, pyrazolyl, triazolyl, 1,2,4-thiadiazolyl, pyrazinyl, pyridazinyl, quinoxalinyl, phthalazinyl, imidazo[1,2-a]pyridine, imidazo[2,1-b]thiazolyl, benzofurazanyl, indolyl, azaindolyl, benzimidazolyl, benzothienyl, quinolinyl, imidazolyl, thienopyridyl, quinazolinyl, thienopyrimidyl, pyrrolopyridyl, imidazopyridyl, isoquinolinyl, benzoazaindolyl, 1,2,4-triazinyl, benzothiazolyl and the like.

The term “benzyl” used herein refers to a phenyl-CH₂— group. Substituted benzyl means a benzyl group in which the phenyl ring is substituted with one or more ring system substituents. Representative benzyl include 4-bromobenzyl, 4-methoxybenzyl, 2,4-dimethoxybenzyl, and the like.

The terms “carboxy” and “carboxyl” used herein refers to a HO(O)C— group (i.e. a carboxylic acid).

The term “carboxyalkyl” used herein refers to a HO(O)C-alkylene-group wherein alkylene is defined herein. Representative carboxyalkyls include carboxymethyl and carboxyethyl.

The term “cycloalkyloxy” used herein refers to a cycloalkyl-O- group wherein cycloalkyl is as defined herein. Representative cycloalkyloxy groups include cyclopentyloxy, cyclohexyloxy, and the like.

The term “hydroxyalkyl” used herein refers to an alkyl group as defined herein substituted with one or more hydroxy groups. Preferred hydroxyalkyls contain lower alkyl. Representative hydroxyalkyl groups include hydroxymethyl and 2-hydroxyethyl.

The term “ring system substituents” used herein refers to substituents attached to aromatic or non-aromatic ring systems inclusive of hydrogen, alkyl, aryl, heteroaryl, aralkyl, aralkenyl, aralkynyl, heteroaralkyl, heteroaralkenyl, heteroaralkynyl, hydroxy, hydroxyalkyl, alkoxy, aryloxy, aralkoxy, acyl, aroyl, halo, nitro, cyano, carboxy, alkoxycarbonyl, aryloxycarbonyl, aralkoxycarbonyl, alkylsulfonyl, arylsulfonyl, heteroarylsulfonyl, alkylsulfinyl, arylsulfinyl, heteroarylsulfinyl, alkylthio, arylthio, nitrile, NO₂ heteroarylthio, aralkylthio, heteroaralkylthio, cycloalkyl, cycloalkenyl, heterocyclyl, heterocyclenyl, aryidiazo, heteroaryldiazo, amidino, Z¹Z²N—, Z¹Z² N-alkyl-, Z¹Z²NCO— or Z¹Z²NSO₂—, wherein Z¹ and Z² are independently hydrogen, alkyl, aryl, and aralkyl, or where the substituent is Z¹Z²N— or Z¹Z²N-alkyl- then one of Z¹ and Z² is acyl or aroyl and the other of Z¹ and Z² is hydrogen, alkyl, aryl, and aralkyl. When a ring system is saturated or partially saturated, the “ring system substituent” further comprises methylene (H₂C═), oxo (O═) and thioxo (S═). Preferred ring system substituents are hydrogen, CF₃, fluoro, alkyl, alkoxy, nitrile or NO₂.

The term “inflammatory disorder” used herein refers to a response to a tissue injury caused by pathogenic microorganisms, trauma, chemicals, toxins, heat, or immune defenses (i.e. autoimmune diseases) involving secretion of several mediators from the injured tissue and induction of immunocytes. When tissue cells are damaged or destroyed, acids and chemical mediators (i.e. cytokines, histamine, bradykinin, serotonin, etc.) get released resulting in the dilation and increased permeability of blood capillaries. Histamine secreted from mast cells or basophiles initiates the response of blood vessels, and serum kinin produced from alpha-2-globulin of blood serum mediates the long-acting response of blood vessels through the blood coagulation mechanism. The blood capillary dilation increases the blood flow, and causes heat and redness. The increased permeability of the blood capillaries cause blood cells, proteins and fluids to exude into surrounding tissues, leading to swelling. Such exudation can accelerate further destruction of cells, and the increased blood pressure stimulates peripheral nerves to cause pain. The pain increases due to secretion of kinin and acids. Other mediators secreted from the tissue include serotonin, prostaglandin, reactants of the complement system, and lymphokine secreted from T-cells.

The inflammatory reaction can occur locally or become systemic. In some cases, pyrogens secreted from bacteria stimulate the thermoregulatory center in the brain and produce a fever. In addition, inflammatory disorders comprise autoimmune disorders which are diseases caused by the body producing an immune response against its own tissues. Autoimmune disorders can be classified into two groups: systemic, causing damage to many organs, and localized, where only a single organ or tissue is directly damaged. Non-limiting examples of autoimmune disorders, with the organ affected include Hashimoto's thyroiditis and Graves' disease (thyroid gland); pernicious anemia (stomach); Addison's disease (adrenal glands); Celiac disease, Crohn's disease, and ulcerative colitis (GI tract); multiple sclerosis and Guillain-Barre syndrome (brain); primary biliary sclerosis, sclerosing cholangitis, and autoimmune hepatitis (liver); and insulin-dependent diabetes mellitus (pancreas). Examples of autoimmune disorders in which multiple organs are affected include, but are not limited to, rheumatoid arthritis, systemic lupus erythematosus (SLE or lupus), Scleroderma, Sjogren's syndrome, Goodpasture's syndrome, Wegener's granulomatosis, and dermatomyositis.

The term “cytokine” as used herein refers to a member of a group of soluble (glyco)proteins released by a cell to send messages and that can act on the same cell (autocrine), on an adjacent cell (paracrine), or on a distant cell (endocrine). Upon binding to a specific receptor, the cytokine causes a change in function or in development of the target cell. Cytokines are involved in reproduction, growth and development, normal homeostatic regulation, response to injury and repair, blood clotting, and host resistance (immunity). Cytokines are produced by many different cell types which modulate the function of other cell types. Cytokines play a role in cellular immune responses as well as inflammatory responses. Cytokines can be classified into five groups: interleukins, colony-stimulating factors, interferons, tumor necrosis factors, and growth factors. Interleukins (IL) is a term used for cytokines produced by leukocytes or macrophage and that act on another leukocyte. Specific interleukins are defined by their number. The main cytokines that mediate inflammation are IL-1, TNF, and IL-8.

Interleukin-1 is produced by macrophages, skin epithelium, fibroblasts, blood vessel endothelium, joint lining cells, cartilage cells, bone-marrow and circulating leukocyte cells, liver cells, adrenal gland cells, pancreatic islet beta cells, fertilized eggs, and cells in the nervous system. IL-1 binds to receptors on a wide variety of cells, including thymus-derived lymphocytes (stimulating immunity), those of the nervous system (causing fever), and endothelial cells (perhaps activating inflammation and blood clotting). Interleukin-6 is also known as beta-2 interferon, IFN-β2, and liver cell growth-stimulating factor, BSF-2, and BCDF. It is produced by a wide variety of cell types, and stimulates the liver to produce proteinase inhibitors and, thereby, dampens inflammation. Interleukin-8 is a group of peptides produced by a variety of cell types. They activate and recruit polymorphonuclear leukocytes in the inflammatory process, and may be involved in initiation of labor and delivery in pregnant women. Interleukin-10 is produced by a subset of helper T cells as well as by B lymphocytes and some cells of the uterus during pregnancy. It inhibits secretion (and function) of cytokine by macrophages and the second population of helper T cells called Th1. The Th1 cells are pro-inflammatory and promote delayed-type hypersensitivity, and the generation of cytotoxic T lymphocytes (antigen-specific killers) and cytotoxic macrophages (nonspecific) in preference to immune responses giving rise to antibodies. Protection against lethal shock triggered by bacterial endotoxin is prevented by suppression of cytokine synthesis.

TNFα and IL-1β are mainly produced from mononuclear leucocytes in response to an endotoxin. They cause increased synthesis of each other and stimulate the production of IL-6, IL-8 and IL-1β. TNFα and IL-1β produce fever, activate the clotting system and mediate inflammation through production of IL-8 and by stimulating expression of adhesion molecules. IL-6 stimulates production of acute phase proteins from the liver and acts to inhibit the production of TNFα and IL-1 β. Expression of inflammatory cytokines from their respective genes is controlled by intracellular transcription factors in particular nuclear factor kappa B (NFκB). NFκB is a primary transcription factor pre-existent in the cellular cytoplasm complexed with the inhibitory subunit IκB. In response to extracellular stimuli, IκB undergoes phosphorylation and ubiquitination allowing its proteosomal degradation. Free NFκB is able to translocate into the nucleus and bind to the promoter region of its target gene

The terms “modifies,” “modified,” and “modulate” are used interchangeably herein and refer to the increase, decrease, elevation, or depression of processes or signal transduction cascades resulting in the altered production or secretion of a protein, peptide, or secondary messenger. The term modifies or modified also refers to the up-regulation or down-regulation of a target gene or a target protein. Non-limiting examples of modifications includes modifications of morphological and functional processes, under- or over production or expression of a substance or substances, e.g., a cytokine, by lymphocytes, failure of cells to produce a substance or substances which it normally produces, production of substances. This modification can result in a variety of disease states.

As used herein, a “therapeutic composition” refers to a composition comprising an active ingredient required to cause a desired effect when a effective amount of the composition is administered to a subject in need thereof.

Within the present invention, an “effective amount” of a composition is that amount of each active component of the therapeutic composition that is sufficient to show a benefit (e.g., a reduction in a symptom associated with the disorder, disease, or condition being treated, or an increase in NO production, decrease in smooth muscle cell proliferation, or decrease in pro-inflammatory and/or cell adhesion molecules). Sample assays to verify the effect are described in the Examples section. When applied to an individual active ingredient, administered alone, the term refers to that ingredient alone. When applied to a combination, the term refers to combined amounts of the active ingredients that result in the benefit, whether administered in combination, serially, or simultaneously.

The term “immune response” as used herein refers to any change in a subject that defends against microorganisms, cancer, disease, or other potentially harmful substances. The response can be cell mediated or antibody mediated and results in change in the production or secretion of cytokines, white blood cells (i.e., neutrophils, lymphocytes (B and T cells), macrophages), chemicals and/or proteins. An immune response includes but is not limited to an inflammatory response, complement mediated, acquired or adaptive immunity, or passive immunity. Immune system disorders occur from inappropriate, excessive, or lacking immune responses. Allergies involve an immune response to a substance that, in the majority of people, the body perceives as harmless. Transplant rejection involves the destruction of transplanted tissues or organs and is a major complication of organ transplantation. Blood transfusion reaction is a complication of blood administration. Autoimmune disorders (such as systemic lupus erythematosus and rheumatoid arthritis) occur when the immune system acts to destroy normal body tissues. Immunodeficiency disorders, such as inherited immunodeficiency and AIDS, occur when there is a failure in all or part of the immune system.

The term “adhesion molecules” or “cell adhesion molecules (CAMs)” as used interchangeably herein, refer to cell surface proteins involved in the binding of cells. The cells, usually leukocytes, can be bound to each other, to endothelial cells, or to extracellular matrix. Specific signals triggered in response to injury and infection control the expression and activation adhesion molecules. Adhesion molecules, following binding to their receptors/ligands, play important roles in the mediation of the inflammatory and immune reactions that comprise one group of the body's defense against insults. Most adhesion molecules characterized so far fall into three general families of proteins: the immunoglobulin (Ig) superfamily, the integrin family, or the selectin family. The members of the Ig superfamily, such as ICAM-1, ICAM-2, ICAM-3, VCAM-1, and MadCAM-1, bind to integrins on leukocytes and mediate their flattening onto the blood vessel wall with their subsequent extravasation into the surrounding tissue. Chemokines such as MCP-1 and IL-8 cause a conformational change in integrins so that they can bind to their ligands. The integrin family act as receptors for the ICAMs and VCAMs. The integrins are heterodimeric proteins consisting of an alpha and a beta chain that mediate leukocyte adherence to the vascular endothelium or other cell-cell interactions.

Different groups of integrins are expressed by different populations of leukocytes providing specificity for binding to different types of adhesion molecules expressed along the vascular endothelium. The selectin family members, L-Selectin, P-Selectin, and E-Selectin, are involved in adhesion of leukocytes to activated endothelium, which is initiated by weak interactions that produce a characteristic “rolling” motion of the leukocytes on the endothelial surface and lead to extravasation through the blood vessel walls into lymphoid tissues and sites of inflammation.

Tissue injury occurs during inflammation and is a progressive process which may eventually lead to organ dysfunction and failure. Circulating neutrophils interact with the vascular endothelium in a three-stage process of rolling, adhesion and migration so that their normally rapid flow through the circulation can be diverted. Leukocyte rolling is mediated through pro-inflammatory cytokines induced expression of selectins on leucocytes and endothelium. Adhesion occurs through binding of leukocyte β2 integrins to endothelial intracellular adhesion molecule-1 (ICAM-1). Expression of adhesion molecules is increased in the most severely ill patients. Adherent leucocytes are then able to migrate into the tissues.

The term “antioxidant” as used herein refers to a substance that, when present in a mixture or structure containing an oxidizable substrate molecule (e.g., an oxidizable biological molecule or oxidizable indicator), significantly delays or prevents oxidation of the oxidizable substrate molecule. Antioxidants can act by scavenging biologically important reactive free radicals or other reactive oxygen species (e.g., O₂⁻, H₂O₂, HOCl, ferryl, peroxyl, peroxynitrite, and alkoxyl), or by preventing their formation, or by catalytically converting the free radical or other reactive oxygen species to a less reactive species. Antioxidants can be separated into two classes, lipid antioxidants, and aqueous antioxidants. Examples of lipid antioxidants include, but are not limited to, carotenoids (e.g. lutein, zeaxanthin, β-cryptoxanthin, lycopene, α-carotene, and β-carotene), which are located in the core lipid compartment, and tocopherols (e.g. vitamin E, α-tocopherol, γ-tocopherol, and δ-tocopherol), which are located in the interface of the lipid compartment, and retinoids (e.g. vitamin A, retinol, and retinyl palmitate) and fat-soluble polyphenols such as quercetin. Examples of aqueous antioxidants include, but are not limited to, ascorbic acid and its oxidized form, “dehydroascorbic acid”, uric acid and its oxidized form, “allantoin”, bilirubin, albumin and vitamin C and water-soluble polyphenols such as catechins, which have high affinity to the phospholipid membranes, isoflavones, and procyanidins.

The term “free radical” as used herein refers to molecules containing at least one unpaired electron. Most molecules contain even numbers of electrons, and their covalent bonds normally consist of shared electron pairs. Cleavage of such bonds produces two separate free radicals, each with an unpaired electron (in addition to any paired electrons). They may be electrically charged or neutral and are highly reactive and usually short-lived. They combine with one another or with atoms that have unpaired electrons. In reactions with intact molecules, they abstract a part to complete their own electronic structure, generating new radicals, which go on to react with other molecules. Such chain reactions are particularly important in decomposition of substances at high temperatures and in polymerization. In the body, oxidized (see oxidation-reduction) free radicals can damage tissues. Antioxidant nutrients (e.g., vitamins C and E, selenium, polyphenols) may reduce these effects. Heat, ultraviolet light, and ionizing radiation all generate free radicals. Free radicals are generated as a secondary effect of oxidative metabolism. An excess of free radicals can overwhelm the natural protective enzymes such as superoxide dismutase, catalase, and peroxidase. Free radicals such as hydrogen peroxide (H₂O₂), hydroxyl radical (HO.), singlet oxygen (¹O₂), superoxide anion radical (O.₂⁻), nitric oxide radical (NO), peroxyl radical (ROO.), peroxynitrite (ONOO⁻) can be in either the lipid or compartments.

The phrase “free radical associated disorder” as used herein refers to a pathological condition in a subject that results at least in part from the production of or exposure to free radicals, for example, oxyradicals, or other reactive oxygen species in vivo. The term “free radical associated disorder” encompasses pathological states that are recognized in the art as being conditions wherein damage from free radicals is believed to contribute to the pathology of the disease state, or wherein administration of a free radical inhibitor (e.g., desferrioxamine), scavenger (e.g., tocopherol, glutathione), or catalyst (e.g., SOD, catalase) are shown to produce a detectable benefit by decreasing symptoms, increasing survival, or providing other detectable clinical benefits in protecting or preventing the pathological state. Examples of free radical disorders include, but are not limited to, ischemic reperfusion injury, inflammatory diseases, systemic lupus erythematosis, myocardial infarction, stroke, traumatic hemorrhage, spinal cord trauma, Crohn's disease, autoimmune diseases (e.g., rheumatoid arthritis, diabetes), cataract formation, age-related macular degeneration, Alzheimer's disease, uveitis, emphysema, gastric ulcers, oxygen toxicity, neoplasia, undesired cell apoptosis, and radiation sickness. Such diseases can include “apoptosis-related ROS” which refers to reactive oxygen species (e.g., O₂) which damage critical cellular components (e.g., lipid peroxidation) in cells stimulated to undergo apoptosis, such apoptosis-related ROS which may be formed in a cell in response to an apoptotic stimulus and/or produced by non-respiratory electron transport chains (i.e., other than ROS produced by oxidative phosphorylation).

The term “oxidative stress” as used herein refers to the level of damage produced by oxygen free radicals in a subject. The level of damage depends on how fast reactive oxygen species are created and then inactivated by antioxidants.

The phrase “inhibiting a condition associated with a lack of or need for nitric oxide (NO)” includes prohibiting, preventing, restraining, and slowing, stopping, or reversing progression, severity or a resultant symptom or effect of the physiological condition. Such conditions include those mentioned in this application, such as pathological platelet aggregation, pathological vasoconstriction, vascular effects of diabetes, stroke, atherosclerosis, hypertension, abnormal vasospasm, and restenosis after angioplasty.

The term “subject” as used herein refers to any living organism in which an immune response is elicited. The term subject includes, but is not limited to, humans, nonhuman primates such as chimpanzees and other apes and monkey species; farm animals such as cattle, sheep, pigs, goats and horses; domestic mammals such as dogs and cats; laboratory animals including rodents such as mice, rats and guinea pigs, and the like. The term does not denote a particular age or sex. Thus, adult and newborn subjects, as well as fetuses, whether male or female, are intended to be covered.

The invention is described in more detail in the following subsections:

I. Oat Species and Extraction Methods

Oats (called Avena in Latin) comprise a group of species that have large, drooping flowerheads and stout, twisted, bent awns growing from the back of the lemmas. Oats are believed to be derived chiefly from two species, wild oat (A. fatua L.) and wild red oat (A. sterilis L.). Oat species with different ploidy levels include but are not limited to: diploids (Avena pilosa, A. clauda, A. ventricosa, A. longiglumis, A. canariensis, A. hirtula, A. wiestii, A. atlantica), tetraploids (A. barbata, A. vaviloviana, A. magna, A. murphyi) and hexaploids (A. fatua, A. occidentalis, A. ludoviciana, A. sterilis).

Avena fatua or wild oat is an introduced species that grows to 80 cm tall. It is an annual with large, open, drooping flowerheads. Wild Oat was introduced from Eurasia. It occurs most often on waste ground, and is a weed in grain fields. In the Columbia Basin region it grows at Creston and Yoho National Park.

Avena sativa or common oat is an introduced species that grows to 80 cm tall. It is an annual with large, open, drooping flowerheads. Common Oat grows on roadsides, railways and waste places. Introduced from Eurasia, it does not persist as an escape from cultivation for more than a year. In the Columbia Basin region it was only collected at Kokanee Glacier Park.

The phenolic compounds can be extracted using known techniques for isolating such compounds from samples of oats, wheat and the like. For example, the phenolic compounds and isomers thereof can be extracted from any oat species, fresh or dried, or parts of oat species, e.g., the hull. Typically, phenolic compounds can be extracted using different solvents. Altering the composition of the solvent can change the extract composition, thus enhancing or reducing the biological activity of phenolic compounds. For example, avenanthramides extracted from oat grain have been described by Canadian patent 1,179,189, which teaches an aqueous steeping method, and U.S. Pat. No. 5,169,660. Isolation of avenanthramides from oat extract are described in Example 1. Three major groups of phytochemicals were separated based on their relative polarity, ranging from most fat soluble, lipophilic, to most water soluble, aqueous. The three groups are alkyl esters, avenanthramides, and oat flavonoids, these phenolics can be separated by liquid chromatography using acidified ethanol.

The isolating the avenanthramides involves the separation of the lipophilic phenolics from the hydrophilic phenolics, which is then further fractionated by double ion exchange, and analyzed by HPLC to yield at least 25 individual compounds. The hydrophilic phenolics can be then further fractionated to yield an avenanthramides fraction, comprising more than 40 different compounds. Identification and quantitation can be performed, for example, by HPLC spectroscopy. The typical oat groat profile consists of three predominant Avenanthramides, A, B and C and a number of isomers and extended chain analogs ((Peterson, D. M. J. Cereal Sci. 33: 115-129 (2001); (Dimberg et al., Cereal Chem., 70(6): 637-641 (1993)). These Avenanthramides can also be made synthetically.

II. Phenolic Compound Compositions

Like all plant foods, oats contain biologically active chemicals or phytochemicals that have been recognized for their health benefits. Oats are also rich in antioxidants, and in particular, phenolic compounds, a class of antioxidants that includes avenanthramides, caffeic acid, ferulic acid, sinapic acid, and cinnamic acid (Collins, J. Agric. Food Chem. 37: 60-66 (1989)). The avenanthramides, a unique source of low molecular weight soluble phenolics present in oats, but not in other cereal grains, exhibit potent antioxidant properties and constitute by far the major phenolic antioxidants present in the oat kernel. Avenanthramides may be useful as antihistaminic, antiallergic, and antiasthmatic drugs, and as inhibitors of lipoxygenase.

In one aspect, the method of the invention relates to providing protection against initiation and development of heart disease, such as atherosclerosis, stroke, and hypertension. The invention can be used to reduce or prevent vascular dysfunction and development of atherosclerotic lesions. This invention can be used to reduce the risk of developing atherosclerosis and hypertension.

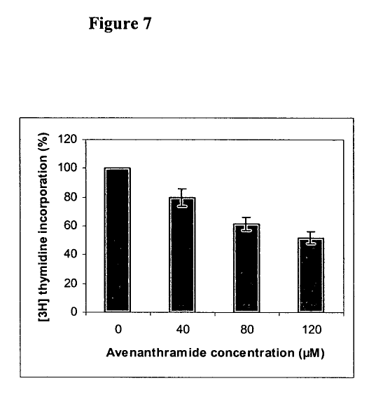

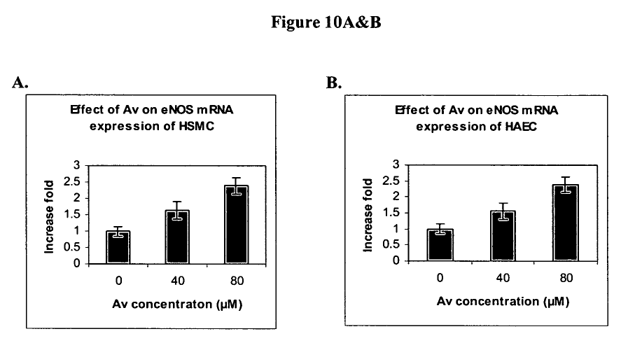

In one embodiment, the invention provides methods of inhibiting vascular SMC proliferation. In another embodiment, the invention provides methods of increasing NO production. The proliferation of intimal vascular smooth muscle cells (SMC) and impaired NO production are both crucial pathophysiological processes in the initiation and development of atherosclerosis. Avenanthramides, including but not limited to, Avenanthramide C (Av-C), can be used to decrease human aortic SMC (HASMC) proliferation and increase NO production (See Example 6 and 7). Av-C can dose-dependently inhibit serum-induced HASMC proliferation as measured by [³H] thymidine incorporation and by counting cell numbers (See FIG. 7). The IC₅₀ of Av-C was around 50 μM. Incubation of cells with 120 μM Av-C for 4 days inhibited cell growth by more than 50%. This inhibitory effect of Av-C was associated with an increase in the expression of p21^{CIP1}, a cyclin-dependent kinase (CDK) inhibitor. In addition, Av-C treatment significantly increased NO production and eNOS mRNA expression as measured by real time PCR (See FIGS. 10A&B). At 80 μM Av-C, NO production and eNOS mRNA expression levels increased by 2.1 and 3.5 fold, respectively. The HASMC expression of vascular cell adhesion molecular-1 (VCAM-1) was not affected by Av-C treatment.

In one aspect, the method of the invention relates to providing protection against free-radical induced disorders by administering phenolic compounds with antioxidant properties. Antioxidants can be characterized in different ways based upon their solubility, their mechanism, or their localization site within the body. Antioxidants can either be fat soluble (lipophilic), water soluble (hydrophilic) or both (Halliwell et al. Arch. Biochem. Biophys. 280:1-8 (1990)). Lipophilic antioxidants, such as carotenoids, can protect the cell membrane and enter the cell to protect other parts of the cell that are surrounded by lipid membranes. However, since it cannot dissolve in the blood, lipophilic antioxidants are transported attached to another molecule. Hydrophilic antioxidants, such as vitamin C, act in the blood. Since they cannot dissolve in the lipid membrane, they must be specifically transported into the cell where it can protect the aqueous parts of the cell. Some antioxidants, such as the avenanthramides, alpha lipoic acid and vitamin E, are both lipophilic and hydrophilic and hence can provide protection almost anywhere in the body. Antioxidants also differ in the class of free radicals (e.g. hydroxyl anion or singlet oxygen) that they can neutralize. For example, vitamin E is effective against peroxyl radicals, singlet oxygen, and peroxynitrite whereas carotenoids only protect against singlet oxygen or peroxyl radicals. Additionally, antioxidants can act as primary antioxidants, which decrease the initiation rate of peroxidation (i.e. transferrin and ceruloplasmin bind prooxidant metal ions) or as secondary antioxidants, which decrease the chain propagation and amplification of peroxidation (i.e. α-tocopherol scavenges oxidizing species). However, most antioxidants are not exclusive, but act with multiple antioxidant properties (e.g., uric acid).

Antioxidants also accumulate in and protect different parts of the body. For example, vitamin C accumulates in the lens of the eye providing protection from cataracts. The carotenoids β-carotene and lutein accumulate in the skin and protect it from the sun's damaging rays. Lutein also accumulates in the macula of the eye, reducing oxidative stress and the risk of macular degeneration. Vitamin E is absorbed into cell membranes, protecting them from oxidative stress. Coenzyme Q10 protects mitochondria from free-radical damage. Some bioflavonoids are thought to be important in protecting the integrity of blood vessels.

The method of the invention can be used to provide protection all over the body, i.e., in both the aqueous compartment and the lipid compartment. In another embodiment, the method of the invention relates to providing protection in a particular compartment, e.g., the lipid compartments or the aqueous compartment.

In another aspect of the invention, the phenolic compounds can be used to protect a subject from increased production or secretion of adhesion molecules that could lead to disease. In a preferred embodiment, avenanthramides could be administered to reduce risk for atherosclerosis or heart diseases. The protective effects of oat extract on inhibiting monocyte-HAEC adhesion is shown in Example 3. The protective effect of oat extract in suppressing expression of adhesion molecules is shown in Example 4.

The methods of the present invention could also be used to treat, reduce, or prevent inflammatory disorders. In another aspect of this invention, the composition could be used to treat or reduce inflammation caused by autoimmune disorders. Methods for producing topical and pharmaceutical compositions, which can be applied to the present invention, are described in U.S. Pat. No. 6,387,398 which is hereby incorporated by reference. The protective effect of oat extract by suppressing pro-inflammatory cytokines is shown in Examples 5.

The methods of the present invention can be used to maintain levels of physiologically acceptable inflammatory molecules in an individual. Administering oat extract can normalize levels of production or secretion of these molecules. Dosing ranges of the oat extract may vary dependent upon preparation. The composition or combination of agents can be administered in amounts sufficient to ensure that the serum level of the phenolic compounds is maintained at an appropriate level or restored or increased to an appropriate level while pro-inflammatory molecules and/or cell adhesion molecules are reduced. The serum levels of the phenolic compounds may be between about 10 μM to 100 mM. Preferably, serum levels of the phenolic compounds may be between about 50 μM to 1 mM. More preferably, serum levels of the phenolic compounds may be 75 μM to 500 μM.

One or more physiologically acceptable phenolic composition can be formulated in a form suitable for topical application. This could be useful for treating skin inflammation due to autoimmune diseases such as Pemphigus folliaceus, pemphigus vulgaris, psoriasis, sarcoidosis, scleroderma, Sjögren's syndrome, rheumatoid arthritis, system lupus erythematosus (SLE), or scleroderma. For example, as a lotion, aqueous or aqueous-alcoholic gels, vesicle dispersions or as simple or complex emulsions (O/W, W/O, O/W/O or W/O/W emulsions), liquid, semi-liquid or solid consistency, such as milks, creams, gels, cream-gels, pastes and sticks, and can optionally be packaged as an aerosol and can be in the form of mousses or sprays. The composition can also be in a sunscreen. These compositions are prepared according to the usual methods. The composition can be packaged in a suitable container to suit its viscosity and intended use by the consumer. For example, a lotion or cream can be packaged in a bottle or a roll-ball applicator, or a propellant-driven aerosol device or a container fitted with a pump suitable for finger operation. When the composition is a cream, it can simply be stored in a non-deformable bottle or squeeze container, such as a tube or a lidded jar. The composition may also be included in capsules such as those described in U.S. Pat. No. 5,063,507.

One or more physiologically acceptable phenolic compound can be administered as compositions by various known methods, such as by injection (subcutaneous, intravenous, etc.), oral administration, inhalation, transdermal application, or rectal administration. Depending on the route of administration, the composition may be coated with a material to protect the compound from the action of acids and other natural conditions which may inactivate the compound. The composition can further include both the avenanthramide compound and another agent, such as a cholesterol-lowering agent.

To administer the composition by other than parenteral administration, it may be necessary to coat the composition with, or co-administer the composition with, a material to prevent its inactivation. For example, the composition may be administered to a subject in an appropriate diluent or in an appropriate carrier such as liposomes. Pharmaceutically acceptable diluents include saline and aqueous buffer solutions. Liposomes include water-in-oil-in-water CGF emulsions as well as conventional liposomes (Strejan et al., J. Neuroimmunol. 7:27 (1984)).

The composition containing at least one avenanthramide may also be administered parenterally or intraperitoneally. In a preferred embodiment, the at least one avenanthramide comprises Av-C. Dispersions can also be prepared in glycerol, liquid polyethylene glycols, and mixtures thereof and in oils. Under ordinary conditions of storage and use, these preparations may contain a preservative to prevent the growth of microorganisms.

Pharmaceutical compositions suitable for injectable use include sterile aqueous solutions (where water soluble) or dispersions and sterile powders for the extemporaneous preparation of sterile injectable solutions or dispersion. In all cases, the composition must be sterile and must be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene gloycol, and the like), suitable mixtures thereof, and vegetable oils. The proper fluidity can be maintained, for example, by the use of a coating such as licithin, by the maintenance of the required particle size in the case of dispersion and by the use of surfactants. Prevention of the action of microorganisms can be achieved by various antibacterial and antifingal agents. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the composition containing the antioxidant in the required amount in an appropriate solvent with one or a combination of ingredients enumerated above, as required. Generally, dispersions are prepared by incorporating the composition into a sterile vehicle which contains a basic dispersion medium and the required other ingredients from those enumerated above.

When the composition containing the antioxidant is suitably protected, as described above, the composition may be orally administered, for example, with an inert diluent or an assimilable edible carrier. The composition and other ingredients may also be enclosed in a hard or soft shell gelatin capsule, compressed into tablets, or incorporated directly into the subject's diet. For oral therapeutic administration, the composition may be incorporated with excipients and used in the form of ingestible tablets, buccal tablets, troches, capsules, elixirs, suspensions, syrups, wafers, and the like. The percentage of the compositions and preparations may, of course, be varied. The amount of active compound in such therapeutically useful compositions is such that a suitable dosage will be obtained.

The tablets, troches, pills, capsules and the like may also contain a binder, an excipient, a lubricant, or a sweetening agent. Various other materials may be present as coatings or to otherwise modify the physical form of the dosage unit. For instance, tablets, pills, or capsules may be coated with shellac, sugar or both. Of course, any material used in preparing any dosage unit form should be pharmaceutically pure and substantially non-toxic in the amounts employed. As used herein “pharmaceutically acceptable carrier” includes any solvents, dispersion media, coatings, antibacterial and antifingal agents, isotonic and absorption delaying agents, and the like. The use of such media and agents for pharmaceutically active substances is well known in the art. Except insofar as any conventional media or agent is incompatible with the active compound, use thereof in compositions of the invention is contemplated.

It is especially advantageous to formulate compositions of the invention in dosage unit form for ease of administration and uniformity of dosage. Dosage unit form as used herein refers to physically discrete units suited as unitary dosages for the subjects to be treated. Each dosage contains a predetermined quantity of active compound calculated to produce the desired therapeutic effect in association with the required pharmaceutical carrier. The specification for the novel dosage unit forms of the invention is dependent on the unique characteristics of the composition containing the antioxidant and the particular therapeutic effect to be achieved. Dosages are determined by reference to the usual dose and manner of administration of the ingredients.

III. Uses

Many disorders or diseases arise due to oxidative stress, the presence of free radicals, and altered immune responses that can lead to inflammatory diseases and cardiovascular diseases. The methods of the present invention can be used to help treat, prevent, or reduce disorders associated with excess free radicals, inflammatory molecules, or adhesion molecules. Examples of such disorders, include, but are not limited to segmental progeria disorders, Down's syndrome; heart and cardiovascular diseases such as atherosclerosis, adriamycin cardiotoxicity, asthma, alcohol cardiomyopathy; cardiovascular disorders such as post-angioplasty restenosis, coronary artery disease, and angina; gastrointestinal tract disorders such as inflammatory & immune injury, diabetes, pancreatitis, halogenated hydrocarbon liver injury; eye disorders such as cataractogenesis, degenerative retinal damage, macular degeneration; kidney disorders such as autoimmune nephrotic syndromes and heavy metal nephrotoxicity; skin disorders such as solar radiation, dermatitis, thermal injury, porphyria: nervous system disorders such as hyperbaric oxygen, Parkinson's disease, neuronal ceroid lipofuscinoses, Alzheimer's disease, muscular dystrophy and multiple sclerosis; lung disorders such as lung cancer, oxidant pollutants (O₃,NO₂), emphysema, bronchopulmonary dysphasia, asbestos carcinogenicity; red blood cell disorder such as malaria Sickle cell anemia, Fanconi's anemia and hemolytic anemia of prematurity; iron overload disorders such as idiopathic hemochromatosis, dietary iron overload and thalassemia; inflammatory-immune injury, for example, glomerulonephritis, autoimmune diseases, rheumatoid arthritis; ischemia reflow states disorders such as stroke and myocardial infarction; liver disorder such as alcohol-induced pathology and alcohol-induced iron overload injury; and other oxidative stress disorders such as AIDS, radiation-induced injuries (accidental and radiotherapy), general low-grade inflammatory disorders, organ transplantation, osteoarthritis, inflamed rheumatoid joints and arrhythmias.

In addition, many disorders or diseases can be attributed to a lack of or need for nitric oxide (NO) including prohibiting, preventing, restraining, and slowing, stopping, or reversing progression, severity or a resultant symptom or effect of the physiological condition. The methods of the present invention can be used to help treat, prevent, or reduce disorders due to a lack of or need for nitric oxide (NO). Such physiological conditions include, pathological platelet aggregation, pathological vasoconstriction, vascular effects of diabetes, stroke, atherosclerosis, hypertension, abnormal vasospasm, and restenosis after angioplasty.

(i) Prophylaxis and/or Treatment

The methods of the invention could be used to prevent or delay onset of disease. For example, people at high risk for cardiovascular disorders could use the purified avenanthramides to reduce their risk. The effect of avenanthramides on smooth muscle proliferation and nitric oxide production of endothelial cells and smooth muscle cells, which play a pivotal role in the initiation and progression of atherosclerosis, was elucidated through the use of the present invention as shown in the Examples.

In one aspect, the methods of the present invention may be used to treat or reduce atherosclerosis. Atherosclerosis is one of the leading cause of morbidity and mortality in western society and is the underlying cause of cardiovascular disease (heart disease and stroke). Overwhelming evidence indicates that inflammatory process plays an important role in the pathogenesis of the disease through the interaction between vascular endothelium and immune cells, in which a variety of mediators including pro-inflammatory cytokines, chemokines and adhesion molecules participate in the initiation and progression of atherosclerosis (Stemme, S. et al. Ann Med. 26: 141-6 (1994)). The effect of oat extract on immune and endothelial cells interactions, which is considered to play a pivotal role in the initiation and progression of atherosclerosis, was elucidated through the use of the present invention as shown in the Examples.

In another aspect, the present invention can be used as a vasodilator due to its increase of the nitric oxide production. Nitric oxide (NO) in the blood exercises various biochemical functions. Nitric oxide serves as an important messenger molecule in the brain and other parts of the body, governing diverse biological functions. In blood vessels, the principal endothelium-derived relaxing factor (EDRF) is believed to be nitric oxide, which stimulates vasodilation. Nitric oxide also inhibits platelet aggregation and is partially responsible for the cytotoxic actions of macrophages. In the brain, nitric oxide mediates the actions of the excitatory neurotransmitter glutamate in stimulating cyclic GMP concentrations. Immunohistochemical studies have localized nitric oxide synthase (NOS) to particular neuronal populations in the brain and periphery. Inhibitors of nitric oxide synthase block physiological relaxation of the intestine induced by neuronal stimulation, indicating that nitric oxide has the properties of a neurotransmitter. In this regard, nitric oxide appears to be a novel type of neuronal messenger, in that, unlike conventional neurotransmitters, nitric oxide is not stored in synaptic vesicles and does not act on typical receptor proteins of synaptic membranes. One function of nitric oxide may be to protect neurons from ischemic and neurotoxic insults. See, Bredt et al., “Cloned and Expressed Nitric Oxide Synthase Structurally Resembles Cytochrome P-450 Reductase,” Nature, Vol. 351, June, 1991, pages 714-718.

In another aspect, the methods of the invention could be used to prevent or delay onset of cardiovascular disease. For example, the reduction of pro-inflammatory cytokines and adhesion molecules has been associated with decreased risk for cardiovascular disease. Both the heart and the blood vessels are sensitive to the effects of pro-inflammatory cytokines as well as vasoactive substances. Nitric oxide is synthesized by inducible nitric oxide synthase (iNOS) in the vascular endothelium and smooth muscle in response to pro-inflammatory cytokines. People at high risk for such disorders could use the purified avenanthramides to reduce their risk.

In another embodiment, the methods of the present invention can be used to enhance resistance to certain disease states. The oxidant/antioxidant balance plays an important role in the pathogenesis of atherosclerosis. The synthesis of pro-inflammatory cytokines, chemokines, and eicosanoids are regulated by redox status through gene activation and post-transcription regulation. Antioxidants can reduce reactive oxygen species and modulate cytokine, chemokine, and eicosanoid production, which may contribute to the potential beneficial effects of antioxidants on reducing the risk of cardiovascular disease.

The methods of the invention can be used to treat immune disorders, such as inflammatory disorders or autoimmune disorders, caused by modified immune responses. In one embodiment, the methods and compositions of the present invention can be used to treat atherosclerosis. In another embodiment, the methods and compositions of the present invention can be used to modulate tumor growth. Significant amounts of IL-8, MCP-1 and IL-6 are produced by endothelial cells, smooth muscle cells and macrophages when they become activated with cytokines or mitogens. IL-8 is a potent chemoattractant to neutrophils, lymphocytes, and basophils and may contribute to recruitment of inflammatory cells in atherosclerotic plaque. IL-8 is produced by the adhesive interaction of endothelial cells and monocytes during the transmigration of monocytes through monolayers. The lymphocytes recruitment to the site of activation in turn increases reactive oxygen species and decreases antioxidant defense. In addition, IL-8 is reported to be chemoattractant for human aortic smooth muscle cell. Further, IL-8 is a potent angiogenic factor which contributes to the growth of atherosclerotic lesions (Simonini et al. Circulation. 101: 1519-1526 (2000)) and tumor growth. MCP-1 is a powerful monocyte chemoattractant, both in vivo and in vitro, and has been shown to be expressed by endothelial cells in early atherosclerotic lesions and involved in monocyte/macrophage recruitment to early lesions. Cushing et al. (Cushing et al. Proc Natl Acad Sci USA. 87: 5134-5138 (1990)) reported that endothelial cells and SMCs of vascular wall upregulated production of MCP-1 when exposed to modified LDL. MCP-1 was also found in macrophage-rich areas of atherosclerotic lesions, but was absent in nonlesional areas. In addition, COX-2 expression and PGE2 is also involved in promoting angiogenesis. In one embodiment, the methods and compositions of the present invention reduce COX-2 expression and PGE2. Thus, the invention can be used to decrease angiogenesis, which can be used in the treatment of tumor growth.

In another embodiment, the methods of the invention can be used to prevent the formation of fatty streaks or atherosclerotic plaques. In addition to monocytes, T lymphocytes are important components in the formation of atherosclerotic plaques. IL-6 has been shown to increase adhesion of circulating lymphocytes, particularly CD4+ to HUVEC and enhanced the expression of ICAM-1, VCAM-1 and E-selectin on endothelial cells. IL-6 is also one of the principal growth-regulatory molecules responsible for the migration and proliferation of SMC. Therefore, this cytokine, together with other chemokines, play a pivotal role in the formation of fatty streaks. Examples 4 and 5 show that incubating HAEC with oat extract reduces the expression of adhesion molecules and of IL-6, respectively.

The methods of the invention may be used to modulate blood lipids. In another embodiment, the methods of the invention may help to reduce CVD risk. It is important to note that Tranilast, [N-(3′4′-dimethoxycinnamoyl)-anthranilic acid], a drug capable of preventing angiographic restenosis after percutaneous transluminal coronary revascularization (Rosanio et al. Thromb Haemost. 82 Suppl 1: 164-170 (1999)), shares a very similar structure with avenanthramide. Tranilast inhibition of restenosis is through the inhibition of vascular smooth muscle cells proliferation and migration (Miyazawa et al. Atherosclerosis. 118: 213-21 (1995); Takahashi et al. Circ Res. 84: 543-50 (1999)) and via inhibition of production of various cytokines from inflammatory cells (Chikaraishi et al. Eur J. Pharmacol, 427: 151-158 (2001)). Oat is unique in the sense of its complement of phenolic compounds, which have powerful antioxidant function associated with the capability of the oxygenated aromatic moiety to scavenge free radicals (Collins, F. W., Quaker Oats Phenolic Antioxidant Study. First Semi-Annual Progress Report, 2000). Avenanthramide are the major phenolic antioxidant identified in oats. Three major Avenanthramides A, B and C are the three major avenanthramides composed of more than 75% of the total antioxidant phenolics present in the avenanthramide fraction, which are closely correlated with antioxidant activity (Emmons et al. J. Agric Food Chem. 47: 4894-8 (1999)).

In another embodiment, the present invention may be effective in the reduction, treatment, or prophylaxis of inflammation and pain, including but not limited to chronic gastritis, arthralgia, benign prostate hyperplasia, chronic and recurrent cystitis, cervical disc, degenerative joint arthritis, rheumatoid arthritis, tennis elbow, osteoportoric pain, migraine, diabetic neuropathy pain and flank pain. Oat extract exhibits high capacity to inhibit adhesive interaction between endothelial cells through inhibition of adhesion molecule expression and to inhibit cytokines and chemokines that are important in the recruitment of immune cells to the site of inflammation.

In yet another embodiment, the methods of the invention can be used to decrease tissue injury and the inflammatory response. Polymorphonuclear leucocytes are one of the main cellular mediators of tissue injury. They accumulate in tissues in response to endotoxin and pro-inflammatory cytokines mediated through IL-8, a powerful chemoattractant and activator of polymorphonuclear leucocytes. Tissue injury occurs due to degranulation of the leucocytes producing proteases (i.e., elastase and matrix metalloproteinases) and the production of reactive oxygen species (ROS). Activated neutrophils produce large amounts of ROS from membrane bound NADPH oxidase which produces the oxygen free radical superoxide and hydroxyl radical. These have been implicated in tissue injury but are also part of the microbial cytotoxic system of the neutrophil. In another aspect of the invention, the alcoholic oat extract and/or avenanthramide can be incorporated into a topical treatment for inflammation that arises due to the allergenic or immune response.

In another embodiment, the invention can be used to treat or reduce respiratory dysfunction. Pulmonary dysfunction can be manifested as tachypnea, hypoxemia and respiratory alkalosis. When severe it may progress to acute lung injury (ALI) and acute respiratory distress syndrome (ARTS). The primary pathological process is pulmonary capillary endothelial dysfunction resulting in interstitial and alveolar edema of protein and phagocytic immune cell rich exudative fluid. Endothelial permeability is increased in response to pro-inflammatory cytokines with progression to alveolar denudation and basement membrane destruction. Neutrophils are sequestrated into the lung in response to IL-8. Concentrations of IL-8 in lung broncheoalveolar lavage fluid in patients with ARDS has been shown to correlate with mortality. In one embodiment, avenanthramides and/or an oat extract composition can be aerosolized and inhaled directly to reduce pro-inflammatory cytokines, such as IL-8.

In another embodiment, the methods of the invention can be used to treat renal dysfunction. Several mechanisms have been proposed for the pathogenesis of acute renal failure. In normal states, the kidney maintains renal blood flow and glomerular filtration through autoregulation dependent on the tone of the afferent and efferent arterioles. The cytokine-induced systemic vasodilatation and relative hypovolemia in diseased states are responsible for renal hypoperfusion. The kidney produces intrinsic vasoconstrictors in response to cytokines and the renin-angiotensin-aldosterone system. In common with other tissues, the kidney is susceptible to leukocyte mediated tissue injury with neutrophil aggregation in response to chemokines and production of proteases and ROSs. In one embodiment, the oat extract composition and/or avenanthramides can be coupled to a delivery vehicle such that the controlled release of the active ingredient is achieved.

Several inflammatory cytokines including interleukin IL-1, tumor necrosis factor (TNF), and interferon produced by activated monocytes and macrophages may stimulate the endothelium to up-regulate genes encoding for chemokines, other cytokines as well as adhesion molecules which mediate attraction and adhesion of immune cells to the endothelium. The upregulated expression of numerous cell surface adhesion molecules is a critical process for the binding of normally non-thrombogenic circulating leukocytes such as the monocytes to the arterial endothelial surface and is one of the earliest detectable events in atherosclerosis. Increased expression of adhesion molecules such as intracellular adhesion molecule-1 (ICAM-1), vascular cell adhesion molecule-1 (VCAM-1), and endothelial leukocyte adhesion molecule-1 (E-selectin) mediate the transmigration of leukocytes to subendothelium leading to the formation of atheromatous plaques. Cytokines are actively involved in leukocyte recruitment, activation and migration. Both activated immune cells and endothelial cells produce a variety of cytokines during the atherogenesis. IL-8 is chemoattractant to T cells and neutrophils, increases proliferation and migration of vascular smooth muscle cells (SMC), and is an angiogenic factor. Monocyte chemoattractant protein-1 (MCP-1), in addition of chemoattractant activity on monocyte and basophils, is a strong cytokine to convert monocyte rolling to firm adhesion on the endothelium expressing E-selectin under flowing conditions. IL-6 is another pro-inflammatory cytokines that can act as a mitogenic stimuli and be responsible for the migration as well as proliferation of SMC.

Dietary factors are known to play significant etiologic roles in the development of atherosclerosis. Diet affects the development of atherosclerosis not only through modulation of lipoprotein metabolism, but also by influencing the inflammatory processes associated with the development of this disease. Production of chemokines and adhesion molecules by endothelial cells has been shown to be regulated by redox sensitive signal transduction and thus may be subjected to modulation by oxidants and antioxidants.

Previous reports indicate that dietary vitamin E, lycopene and polyphenolics with antioxidant activity inhibit adhesion of immune cells to endothelial cells through modulation of cytokines production, adhesion molecules expressions, and chemokines release in vivo and in vitro. Consumption of oat is associated with reduced risk of coronary heart disease and has been shown recently to improve endothelial dysfunction (Katz, D. L. et al. Prev Med. 33: 476-84 (2001)). Like all monocot cereals, oats (Avena sativa L.) contain a number of phytochemicals containing a phenolic moiety with free-radical scavenging capability and thus exhibiting antioxidant properties in vitro.

The active interaction of the endothelium with cells of the immune system is widely acknowledged, particularly as it relates to inflammatory processes and foam cell formation in the development of atherosclerosis. Stimulation of either cell type, viz., human arterial endothelial cells (HAEC) or monocytes, results in expression of numerous adhesion molecules and/or counter-ligands, as well as induces further secretion of other pro-inflammatory factors, including cytokines and chemokines. As this process continues, the monocytes, which typically roll, are triggered, activated to bind strongly, and stick to the endothelium, eventually infiltrating where plaque formation is initiated. Stimulation of HAEC and/or monocytes of human origin represents an effective model for assessing the early events of vascular modification and atherogenesis. Enrichment of HAEC or monocytes with antioxidants, (i.e., vitamin E, probucol, N-acetylcysteine, and pyrrolidine dithiocarbamate) decreases adhesion and interaction of EC with immune/inflammatory cells.

The methods of the invention can be used to reduce the production or secretion of cell adhesion molecules. In a preferred embodiment, the methods of the invention can be used to reduce fatty streaks that may lead to diseased states. Cell adhesion molecules (CAM) play an important role in the development of the early fatty streaks and fibrous atherosclerotic plaques. VCAM-1 expressed by endothelial cells, through binding to VLA-4 integrin mediates the adhesion of leukocytes to activated endothelium. ICAM-1 through binding to LFA-1 and MAC-1 integrins is involved in the adhesion of leukocytes and neutrophils to the endothelium during activation, flattening, and extravasation. Increased expression of ICAM-1 in the atherosclerotic plaques has been demonstrated by the presence of an increased immunoreactivity to ICAM-1 antibody. The increased E-selectin expression in endothelial cells surface induced by cytokines, bacterial toxins, and oxidants also mediate immune/endothelial cell adhesions. While ICAM-1 is constitutively expressed by the normal unstimulated endothelial cells, the expression of VCAM-1 and E-selectin is negligible.

The methods of the invention can be used to decrease cell expression of adhesion molecules, production of chemokines and pro-inflammatory of cytokines. Endothelial cells do not normally express elevated levels of adhesion molecules and thus, do not normally support excessive attachment to leukocytes. However, such an interaction is stimulated by exposure to a number of stimuli, including LDL, oxidized LDL, bacterial lipopolysaccharide, and inflammatory cytokines such as IL-1β, which induces phenotypic changes. The cytokine- and/or chemokine-stimulated adhesion of monocytes to endothelium of bovine, porcine, and human origin has been routinely used as a model to investigate interactions between leukocytes and the endothelium. Pretreatment of HAEC with oat extracts dose-dependently reduced IL-1β stimulated HAEC expression of adhesion molecules, productions of chemokines and pro-inflammatory of cytokines and their adherence to U937 cells as shown in Examples 3-5. The effect of enrichment of human aortic endothelial cells (HAECs) on their adherence to monocyte and production of inflammatory cytokines and chemokines is shown in Examples 3, 4, and 5

(ii) Prevention

In one aspect, the methods of the invention can be used to reduce the risk, prevent the disorder or delay onset of cardiovascular disorders. The method of the invention can be used to reduce the risk, prevent or delay onset of heart disease. In one embodiment, the compounds of the present invention can be incorporated into polymers, such as those used to make cardiovascular stents, or used as a coating on stents to prevent proliferation of SMC and prevent restenosis after angioplasty. The compounds of the present invention can, for example, be combined with and/or impregnated into polymers (e.g., biodegradable polymers, slow release polymers, and/or controllable or inducible-release polymers) such that the compound can be delivered to the target site over time. The polymer can be impregnated with one or more compound of the present invention such that release can be controlled and directed to the target area (e.g., vascular cells). In addition, the stents can comprise one of more compounds of the present invention combined with other compounds (e.g., antioxidants, such as vitamin E, and other phenolic compositions) to provide synergist effects and/or with other drugs (e.g., antibiotics, growth factors, cholesterol reducing agents, such as statins, anti-neoplastics, immunosupressives, migration inhibitors, and enhanced healing factors) to reduce the risk of restenosis and/or intimal hyperplasia.

In another aspect, the methods of the invention can be used to reduce the risk, prevent the disorder or delay onset of oxidative stress disorders. The method of the invention may have uses in endurance exercise training by protecting against oxyradical increase. The method of the invention could have applications in vascular biology such as reducing oxidative stress or inhibit foam cell production. In another embodiment, the method of the invention could be used to improve vision as the avenanthramides could be transported to the eye lens and macular and reduce cataract formation. In yet another embodiment, the method of the invention could have uses in neuroscience by affecting signal transduction pathways.

The method of the invention can be used to reduce the risk, prevent or delay onset of inflammatory disorders and/or heart disease. Leukocyte adhesion to the endothelium is a multi-stage process occurring early in the pathogenesis of atherosclerosis and inflammation. In a preferred embodiment, the invention can be used to prevent onset of the disease. In response to infection and tissue injury, endothelial cells become activated and express molecules through the generation of vasoactive compounds, lipid-based activators, chemokines, and specific cell surface adhesion molecules. Leukocyte adhesion to the endothelium is facilitated by the presence of leukocyte adhesion molecules or receptors. Intercellular adhesion molecule-1 (ICAM-1) is expressed by resting endothelial cells. Conversely, endothelial-leukocyte adhesion molecule-1 (ELAM-1) and vascular cell adhesion molecule-1 (VCAM-1) are normally absent in resting endothelial cells, but their expression can be induced by various plasma components, including cytokines such as interleukin-1β. LDL cholesterol, cytokines, primarily IL-1β and tumor necrosis factor (TNF), nitric oxide, and oxidized lipoproteins modulate the adhesiveness of the endothelium via stimulating adhesion molecule expression. The similarities between inflammation and atherogenesis suggest a role for ROS in these processes. In one embodiment, the methods of the invention can be employed to increase cellular accumulation of antioxidant compounds in the endothelium which can reduce the expression of adhesion molecules by quenching free radical production. In fact, increased expression of adhesion molecules, such as VCAM, and marked enhancement of monocyte adhesion after stimulation of human umbilical vein endothelial cells with IL-1β were both significantly inhibited after preincubation of monolayers with the antioxidants.

In one embodiment, the methods of the present invention can also be used in food supplements and nutraceutical formulations. A nutraceutical refers to formulations of natural or naturally-derived agents that can impart medical and/or health benefits. Thus, oat extract and/or phenolic compositions described in this invention can be incorporated into supplements or formulated as nutraceutical supplements themselves. In addition, foodstuff can be enhanced with phenolic compositions, such that the enhanced foodstuff can delay onset of inflammatory disorders and/or heart disease. In another embodiment, a composition comprising of an alcoholic extract of oats can be utilized as a nutraceutical supplement. In a preferred embodiment, the supplement would comprise purified avenanthramides.

This invention is further illustrated by the following examples which should not be construed as limiting. The contents of all references, patents and published patent applications cited throughout this application, are incorporated herein by reference.

### EXAMPLES

The following experiments were performed to establish methods for increasing NO, inhibiting smooth muscle cell proliferation, inhibiting a physiological condition associated with a lack of or need for nitric oxide (NO), and reducing pro-inflammatory molecules and cell adhesion molecules through the administration of the phenolic compositions of the present invention.

### Example 1

### Materials and Methods

(i) Cell Culture

HAEC were purchased from Clonetics Laboratories (San Diego, Calif.) and cultured in MCDB-131 medium (Sigma Chemical, St. Louis, Mo.). Passages 6-8 were used in this study. The culture medium contained 2% fetal bovine serum (FBS) (Gibco, Grand Island, N.Y.), 2 mmol/L L-glutamine (Gibco), 100 U/mL penicillin (Gibco), 100 U/mL streptomycin (Gibco), 1 ug/mL hydrocortisone, 0.01 ug/mL epidermal growth factor (EGF), 0.5 mL bovine brain extract (BBE), 0.5 μg/mL amphotericin B (Sigma). RACE were seeded in the 1% gelatin (Sigma) coated T-75 flasks, 24-well plates and 96-well plates. The medium was changed every other day until the cells grew to confluence. 6-8 passage cells were employed and experiments were conducted in triplicate or quadruplicate. U937 cells (American Type Culture Collection, Rockville, Md.) grew in suspension culture in RPM1-1640 medium (Life Technologies, Grand Island, N.Y.) supplemented with 10% FBS, 2 mmol/L L-glutamine, 100 U/mL penicillin, 100 mg/mL streptomycin.

Human Aortic Smooth Muscle Cells (HSMC) are derived from tunica intima and tunica media of normal human, fibrous plaque-free aorta. They are cryopreserved at second passage and can be cultured and propagated at least 16 population doublings. HSMC respond to various factors by proliferating or differentiating (Fager, G. et al, In Vitro Cell. Biol. 25(6):511 (1989); Hoshi, H. et al, In Vitro Cell. Biol. 24(4):309 (1988)). They are a well established cell system for the study of human vascular disorders such as atherosclerosis and stroke (Orekhov, A. N. et al, Lab. Invest. 48:749 (1983)); Jonasson, L. et al, Arteriosclerosis 6(2):131 (1986)).

(ii) Preparation of Oat Avenanthramides

A hulless, “identity preserved” variety of oats can be used. To obtain an enrichment of avenanthramides dry milling can be employed, as described by Gray et al. (Gray et al. “Enrichment of Oat Antioxidant by Dry Milling and Sieving,” J Cereal Sci. 32: 89-98 (2000)).

(iii) Extraction and Preparative Purification of the Avenanthramide Fraction

The pearlings obtained by dry milling can be added to refluxing acidified ethanol and stirred vigorously. The stirring mixture can be removed from the heat, cooled, then centrifuged. The resulting supernatant can be decanted through a filter to give a clear greenish-yellow extract. The insoluble pellet can be re-suspended in the same solvent and the above extraction procedure can be repeated for further purification.

To remove non-phenolic, lipophilic components from this micella, hydrophobic resin can be added and the mixture can be concentrated to dryness in vacuo at 35° C. by rotary evaporation. The dried mixture can be suspended in acidified 30% ethanol and quantitatively transferred to a graduated glass chromatography column containing hydrophobic resin, gravity packed and pre-equilibrated in acidified 30% acidified ethanol. The volume can be eluted with acidified 30% ethanol followed acidified 50% ethanol. The combined eluates can be concentrated to a syrup in vacuo at 35° C. by rotary evaporation. The syrup can be taken up in acidified 30% ethanol and chromatographed on a similar column. The column can be eluted again and the eluate concentrated to a syrup in vacuo at 35° C. by rotary evaporation.

The de-fatted extract can be further purified to remove flavonoids, benzoic and cinnamic acids and polar, non-phenolic components by double chromatography. The absorbed avenanthramides, flavonoids and phenolic acids can be recovered by eluting the column with ethanol and concentrating the eluate to dryness in vacuo at 35° C. by rotary evaporation. The dry residue can be taken up in acidified 50% ethanol and loaded onto a second size exclusion column. The column can be eluted with acidified 50% ethanol to remove the flavonoids and phenolic acids and the absorbed avenanthramides recovered with 95% ethanol. This purified “avenanthramide fraction” can be freeze-dried to a deep orange powder after concentration to a small volume in vacuo at 35° C. by rotary evaporation and dilution with water. Various methods for obtaining such extracts are known in the art. Non-limiting examples include Dimberg Häll et al. (Cereal Chemistry 70: 637-641 (1993)), Dimberg et al. (J. Cereal Science 24: 263-272 (1996)), Xing et al. (JAOCS 74(3): 303-307 (1997)), Collins et al. (J. Chromatography 445: 363-370 (1988)), Collins (J. Agric. Food Chem 37: 60-66 (1989)), and U.S. Pat. No. 5,169,660, which are herein incorporated by reference.

(iv) Avenanthramide Analysis

Total and individual avenanthramides can be determined by analytical HPLC by known methods. Individual avenanthramides can be identified by comparison of relative retention times and UV spectra with authentic standards. Quantification can be achieved by comparing peak areas with an external Avenanthramide A standard at 330 nm and expressed as Avenanthramide A weight equivalents.

(v) Oat Extract Supplementation

Oat extract stock solution (100 mg/mL) was made in dimethyl sulphoxide (DMSO). The DMSO-oat extract solution was then diluted by MCDB-131 medium to make final concentrations of 4, 20 and 40 μg/mL to supplement HAEC. The concentration of DMSO in cultures media was 0.04%, which is tested to be not toxic to the cells (data not shown). Confluent monolayer HAEC in 24-well plates and 96-well plates were incubated with different concentrations of oat extract at 37° C. for 24 h.

(vi) Oat Extract Cytotoxicity

The oat extracts were dissolved in DMSO and diluted into MCDB-131 medium in different concentrations and incubated with HAEC for 24 h. The cytotoxicity of oat extracts at 4 μg/mL and 40 μg/mL in 0.04% DMSO (final concentration in medium) was tested using Trypan blue exclusion test.

(vii) Synthesis of Avenanthramide C (Av-C)

Avenanthramide-C (Av-C) can be synthesized, for example, by the techniques described in Peterson, D. M. J. Cereal Sci. 33: 115-129 (2001).

(viii) Fluorescent Labeling of Monocytes

U937 cells (American Type Culture Collection, Rockville, Md.), a human monocytic cell line, were used for monocyte-endothelial cell adhesion assay. This cell line has been used as a model for the blood-born monocyte in endothelial cell adhesion experiments. This cell line exhibits many characteristics of monocytes and is easy to use. A virtually unlimited number of cells can be prepared and are relatively uniform. This cell line has been an important tool in the investigation of the mechanisms involved in monocyte-endothelium attachment (DiCorleto et al. J Clin Invest. 75: 1153-1161 (1985)). The U937 cells were fluorescently labeled by incubating the cells (1×10⁷ cells/5 mL) with 5 μmol BCECF-AM/L [2′,7′-bis-(2-carboxyethyl)-5-(and-6)-carboxy-fluorescein acetoxymethyl ester BCECF-AM (Molecular Probes, Eugene, Oreg.)] in PRM1-1640 medium for 30 min at 37° C. and 5% CO₂. BCECF-AM is a non-fluorescent lipophilic compound in which the ester bound is cleaved by intracellular esterase and becomes a highly charged fluorescent BCECF that is retained by viable cells. The BCECF-AM was prepared as a 1 g/L stock in DMSO and was stored at −80° C. After labeling, the cells were washed 3 times with 1% FBS in PBS to remove excess dye. Finally, cells were resuspended in MCDB-131 medium at a density of 5×10⁸ cells/L for the quantitative adhesion assay (Vaporciyan et al. J Immunol Methods. 159: 93-100 (1993)).

(ix) U937 Cell Adhesion Assay

HAEC were cultured to confluence in 24-well plates and were treated with varying concentrations of oat extract for 24 hr. HAEC were then washed with PBS and activated with IL-1β (Endogen, Woburn, Mass.) 5 ng/mL for 6 h. BCECF-labeled U937 cells (1×10¹ were incubated with HAEC for 30 min at 37° C. After incubation, nonadherent cells were removed by washing each well 3 times with 1% FBS-PBS. The attached cells were lysed with 0.5 mL of 50 mmol/L tris buffer (pH 7.6) containing 0.1% sodium dodecyl sulfate. The fluorescence intensity of each well was measured with a Cytofluor (PerSeptive Biosystems, Framingham, Mass.) fluorescence multiwell plate reader set at excitation and emission wavelengths of 485 nm and 530 nm, respectively. With each set of experiments, a separate plate containing known numbers of U937 cells labeled with BCECF-AM was prepared for determination of a standard curve of fluorescence units per cell.

(x) Adhesion Molecule Expression

Confluent HAEC in 96-well plates (Becton Dickinson Labware, Franklin Lakes, N.J.) were incubated with or without oat extract (4, 20 and 40 μg/mL) at 37° C. for 24 h. After the cells were washed with PBS, 5 ng/mL IL-1β (Endogen) was added to stimulate the cells at 37° C. for 6 h. Following decanting the medium, the cells were fixed with 1% paraformaldehyde at room temperature for 30 min. Enzyme linked immunosorbent assay (ELISA) was used to measure adhesion molecule expression. The plates were washed with PBS, blocked with 10% FBS for 1 hr. Monoclonal antibodies against human ICAM-1, VCAM-1 and E-selectin (PharMingen, San Diego, Calif.) were added at 2, 5 and 5 μg/mL respectively in 10% FBS/PBS for 1 h at room temperature. The secondary antibody, horseradish-peroxidase-conjugated anti-mouse IgG (Santa Cruz, Calif.) was added at 1:1000 dilution and incubated at room temperature for 1 hr. This was followed by addition of horseradish peroxidase substrate (Bio-Rad, Hercules, Calif.) and incubation for 1 hr. The plates were read at OD 405 nm by a plate reader (Bio-Tek Instruments, Winooski, Vt.).

(xi) Cytokine Expressions of HAEC

Confluent HAEC in 24-well plates (Becton Dickinson Labware) were incubated without or with oat extract (4, 20 and 40 μg/mL) at 37° C. for 24 h. After the cells were washed with PBS, 5 ng/mL IL-1β (Endogen) was added to stimulate the cells at 37° C. for 24 hr. After centrifugation, the supernatants were collected and stored at −80° C. Sandwich ELISA was used to measure IL-6, IL-8 and MCP-1 expressions. Pro-Bind 96-well plates (Becton Dickinson) were coated with capture antibodies for IL-6, IL-8 (R&D System, Minneapolis, Minn.) and MCP-1 (PharMingen) overnight. After blocking with 10% FBS (Gibco) in PBS for 2 hr, the standards and samples were added and incubated at room temperature for 2 hr. Then biotinylated antibodies for IL-6, IL-8 (R&D System), MCP-1 (PharMingen) were added for 2 hr at room temperature. Avdin-peroxidase (Sigma) was used to amplify the reaction. Finally, horseradish peroxidase substrate (Bio-Rad) was added and incubated for 1 hr. The plates were read at OD 405 nm in a plate reader (Bio-Tek Instruments).

(xii) Statistical Analysis

Data were analyzed using SYSTAT statistical package version 8.0 (Systat, 2001). Multiple comparisons of means were conducted by Tukey Post Hoc test. The overall oat extract effect was determined by ANOVA. Values are presented as means±SD and p<0.05 is considered significant.

### Example 2

### Cytotoxicity Test

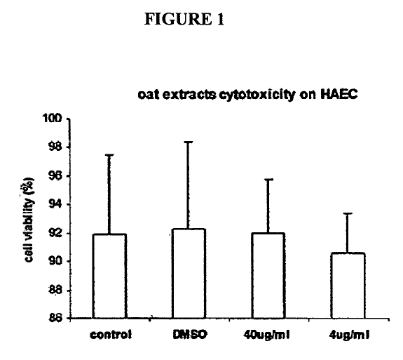

FIG. 1 shows oat extracts and DMSO cytotoxicity on HAEC. Confluent human aortic endothelial cells (HAEC) were incubated with 0, 4 and 40 μg/mL oat extracts and 0.04% DMSO for 24 h at 37° C. Cytotoxicity was measured by Trypan blue exclusion test. Data are the mean±SD of 3 experiments, each performed in triplicate. *p<0.05, **p<0.01 compared with control. Oat extract had no cytotoxicity on HAEC up to the 40 μg/mL concentration tested. 0.04% DMSO in MCDB-131 medium solution showed also no toxicity on HAEC during 24 hr incubation.

### Example 3

### Effect of Oat Extract on Monocyte-HAEC Adhesion

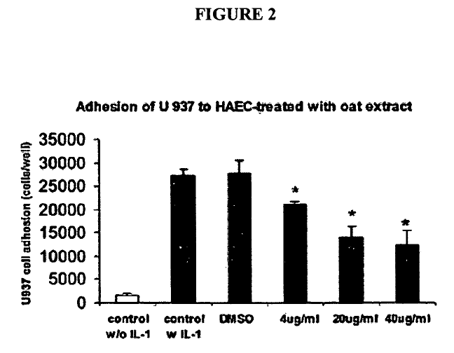

The effect of oat extracts on monocyte-endothelial cell adhesion is shown in FIG. 2. Confluent human aortic endothelial cells (HAEC) were incubated with 0, 4, 20 and 40 μg/mL oat extracts for 24 h at 37° C. The HAEC were then stimulated by interleukin (IL)-1β (5 μg/mL) at 37° C. for 6 h. A total of 10⁷ U937 cells were added onto HAEC and incubated at 37° C. for 30 min. The adhesion of U937 cells to HAEC was determined as described in Example 1. Data are the mean±SD of 3 experiments, each performed in triplicate. *p<0.05, **p<0.01 compared with control. There was trivial adhesion of U937 to HAEC without IL-1β stimulation. Pre-treatments of HAEC with oat extracts or DMSO contributed little to that basal adhesion (data not shown). However, when HAEC was stimulated with 5 ng/mL IL-1β for 6 h, their adherence to U937 cells increased (p<0.01) (FIG. 2). Pretreatment of HAEC with oat extracts for 24 h before activation with IL-1β significantly reduced their adherence to U937 cells (p<0.05). Pretreatment of IL-1β-stimulated HAEC with 4, 20 and 40 μg/mL oat extracts reduced their adherence to U937 cells by 20%, 40% and 45%, respectively (FIG. 2).

### Example 4

### Effect of Oat Extract on the Expression of Adhesion Molecules

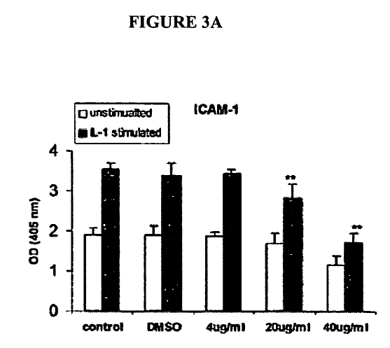

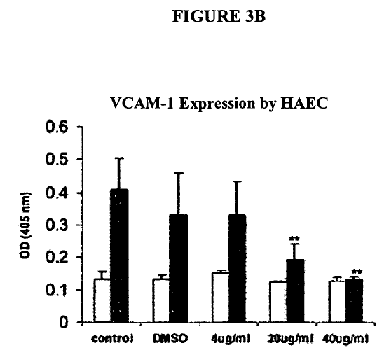

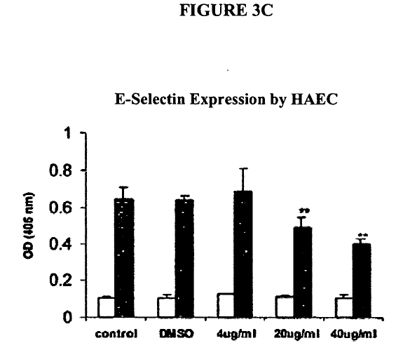

The effect of oat extracts on HAEC expression of adhesion molecules is shown in FIG. 3. Confluent human aortic endothelial cells (HAEC) were incubated with 0, 4, 20 and 40 μg/mL oat extracts for 24 h at 37° C. The HAEC were then stimulated by interleukin (IL)-Iβ (5 ug/mL) at 37° C. for 6 h. The expression of ICAM-1, VCAM-1 and E-selectin (A, B and C, respectively) on the cell surface was measured using EL1SA as described in Example 1. Data are the mean±SD of 3 experiments, each performed in triplicate. *p<0.05, **p<0.01 compared with control. The unstimulated HAEC constitutively expressed ICAM-1 was greater than VCAM-1 and E-selectin expressions (FIG. 3). With 5 ng/ml IL-1β stimulation for 6 h, the expression of adhesion molecules on the surface of HAEC increased significantly (P<0.01) (FIG. 3). Oat extract didn't significantly affect constitutive expression of adhesion molecules but significantly (P<0.05) inhibited IL-1β-stimulated expressions of ICAM-1 (FIG. 3A), VCAM-1 (FIG. 3B) and E-selectin (FIG. 3C). Pretreatment HAEC with 20 μg/mL and 40 μg/mL oat extracts significantly reduced the expressions of adhesion molecules on IL-1β-stimulated HAEC compared with the untreated cells (P<0.05). Pretreatment with 40 μg/mL oat extract resulted in the most effective inhibition of adhesion molecules expression. The expressions of ICAM-1 and VCAM-1 in the IL-1β-stimulated HAEC were nearly abrogated by preincubation of HAEC with 40 ug/mL oat extracts (FIG. 3).

Following IL-1β stimulation of HAEC, the expressions of ICAM-1 and VCAM-1 increased several folds and pretreatment of HAEC with oat extracts dose-dependently inhibited the expressions of these adhesion molecules leading to a decreased adhesion of U937 cells to IL-1β-stimulated HAEC. The highest dose of oat extracts 40 μg/mL totally abrogated the stimulatory effect of IL-1β.

The study shows, oat extracts inhibited IL-1β stimulated HAEC adhesion to U937, but not basal unstimulated adhesion. It was also found that IL-1β-activated HAEC expressions of three important adhesion molecules was inhibited while the constitutive expression was unaffected by oat extracts treatment. Therefore, the reduction of HAEC adhesion to U937 by oat extracts appears to be mediated by the suppression of adhesion molecules expressions by HAEC. The secretions of IL-8, MCP-1 and IL-6 by HAEC were also affected by oat extracts treatment suggesting that oat extract may interfere with some common activation mechanism responsible for the induction of these active molecules. Although the effect of oat extract on NF-κB inhibition was not examined, the results suggest oat extract treatment may have influenced this signaling pathway in HAEC. It is well established that the nuclear transcription factor NF-κB binding sites in the promoters of ICAM-1, VCAM-1 and E-selectin genes are necessary for their induction via cytokines (Whelan et al. Nucleic Acids Res. 19: 2645-53 (1991); Collins et al. Faseb J. 9: 899-909 (1995)). Activation of NF-κB also results in expression of mRNA of a variety of pro-inflammatory mediators such as IL-8, IL-6, MCP-1 and adhesion molecules (Christman et al. Intensive Care Med. 24: 1131-8 (1998)). Because some evidence support the role of ROS in NF-κB activation (Schreck et al. Free Radic Res Commun. 17: 221-237 (1992)), antioxidants have been investigated as inhibitor of NF-κB activation. Direct treatment with oxidants such as H₂O₂ activated NF-κB. While antioxidants have been shown to inhibit NF-KB activation in vitro (Van den Berg et al. Br J Nutr. 86 Suppl 1: SI21-7 (2001)). Up-regulation of endogenous oxidant defenses has been demonstrated to suppress NF-κB activation (Mirochnitchenko et al. J Immunol, 156: 1578-1586 (1996)). Activation of NF-κB in vitro has been shown to be inhibited by a variety of antioxidants including vitamin E derivatives, pyrrolidine dithiocarbamante (PDTC), N-acetylcysteine (NAC), ascorbic acid and etc. (Christman et al. Intensive Care Med. 24: 1131-1138 (1998)). Therefore, the mechanism for antioxidant inhibition of pro-inflammatory cytokines, chemokines, adhesion molecules and HAEC adhesions to monocyte potentially were mediated by inactivation of NF-κB signaling pathway.

### Example 5

### Effect of Oat Extracts on the Production of Cytokines

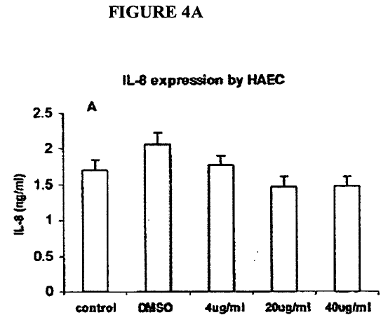

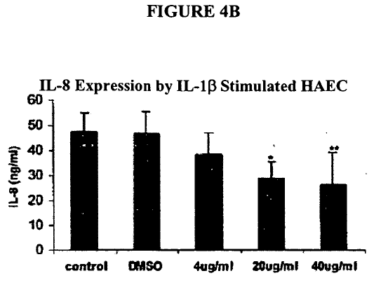

The effects of oat extracts on HAEC expression of IL-8, IL-6, and MCP-1 are shown in FIGS. 4, 5, and 6, respectively. FIG. 4 shows the effect of oat extracts on HAEC expression of IL-8. Confluent human aortic endothelial cells (HAEC) were incubated with 0, 4, 20 and 40 μg/mL oat extracts for 24 h at 37° C. The HAEC were then stimulated by interleukin (IL)-Iβ (5 μg/mL) at 37° C. for 24 h. The expression of IL-8 on the cell surface was measured using ELISA as described in Example 1. Blank bars shown in FIG. 4A represent constitutive expressions. Shaded bars shown in FIG. 4B represent IL-1β stimulated expressions. Data are the mean±SD of 3 experiments, each performed in triplicate. *p<0.05, **p<0.01 compared with control.

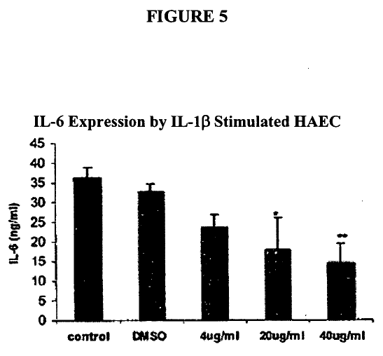

FIG. 5 shows the effect of oat extracts on HAEC expression of IL-6. Confluent human aortic endothelial cells (HAEC) were incubated with 0, 4, 20 and 40 μg/mL oat extracts for 24 h at 37° C. The HAEC were then stimulated by interleukin (IL)-1β (5 μg/mL) at 37° C. for 24 h. The expression of IL-6 on the cell surface was measured using ELISA as described in Example 1. Shaded bars shown in FIG. 5 represent IL-1β stimulated expressions. The IL-6 baseline was too trivial to be detected. Data are the mean±SD of 3 experiments, each performed in triplicate. *p<0.05, **p<0.01 compared with control.

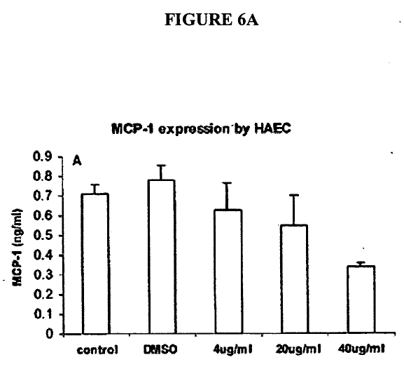

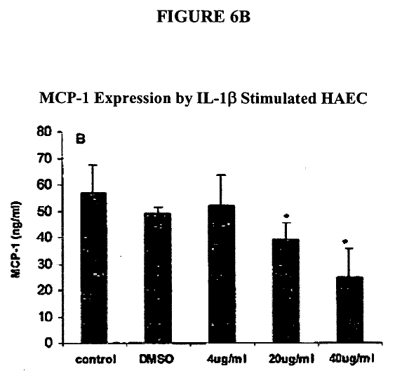

FIG. 6 shows the effect of oat extracts on HAEC expression of MCP-1. Confluent human aortic endothelial cells (HAEC) were incubated with 0, 4, 20 and 40 μg/mL oat extracts for 24 h at 37° C. The HAEC were then stimulated by interleukin (IL)-Iβ (5 μg/mL) at 37° C. for 24 h. The expression of MCP-1 on the cell surface was measured using ELISA as described in Methods. Blank bars shown in FIG. 6A represent constitutive expressions. Shaded bars shown in FIG. 6B represent IL-1β stimulated expressions. Data are the mean±SD of 3 experiments, each performed in triplicate. *p<0.05, **p<0.01 compared with control.

The production of IL-8, IL-6 and MCP-1 by HAEC significantly increased with IL-1β stimulation compared with unstimulated cells (P<0.01). There was no detectable levels of IL-6 baseline, and very low level of IL-8 and MCP-1 were present before activation of HAEC with IL-1β (FIGS. 4A & 6A). Supplementation of HAEC with oat extracts showed no effect on the IL-8 and IL-6 expressions in unstimulated cells. However, pretreatment of unstimulated HAEC with 40 μg/mL oat extract significantly inhibited MCP-1 production (P<0.05) (FIG. 6A). Pretreatment HAEC with oat extract 20 μg/mL and 40 μg/mL decreased the production of IL-8 (FIG. 4B), IL-6 (FIG. 5) and MCP-1 (FIG. 6B) (P<0.05) following stimulation with IL-1β.

In a confluent HAEC monolayer culture, the constitutive expression of IL-8 is very low, however, its expression is increased by 30 fold following the activation of HAEC. Supplementing HAEC with oat extracts dose-dependently inhibited IL-8 production in the IL-1β activated HAEC.

The production of MCP-1 by unstimulated HAEC in the present study was low. However it was remarkably increased with IL-1β stimulation (FIGS. 6A & B). Supplementing HAEC with oat extracts dose-dependently inhibited the MCP-1 production by the activated cells. However, only highest dose of oat extract (40 μg/mL) was effective to reduce its production to a significant level in the inactivated cells. Monocyte recruitment to the site of inflammation is believed to be a major determinant of atherosclerotic lesion size and complexity (Reape et al. Atherosclerosis. 147: 213-25 (1999)). Thus, the findings that oat extracts dose-dependently decreased MCP-1 production by IL-1β stimulated HAEC may have important implications in terms of the potential role of oat in reducing recruitment and transmigration of monocytes across the endothelium.

In the HAEC monolayer, the basal production of IL-6 was too low to be detected. However, with IL-1β stimulation for 24 hr, the production of IL-6 by HAEC was increased significantly, and pre-incubation of HAEC with oat extracts dose-dependently reduced the IL-6 production in IL-1β stimulated HAEC. Therefore, suppression of IL-6 production in activated HAEC by oat extracts provides another potential protective mechanism by which consumption of oat may contribute to the reduction of risk of atherosclerosis, through inhibiting SMC proliferation, migration and atherosclerotic plaque formation.

Recruitment of monocytes to the intima is one of the earliest events in the formation of an early lesion of atherosclerosis (Libby, P. J Intern Med. 247: 349-358 (2000)). In response to inflammatory stimuli such as IL-1β, TNF-α, the activated endothelium recruits leukocytes to the site of activation by production of chemokines such as IL-8, MCP-1 and IL-6, then selectively expresses adhesion molecules to capture the immune cells (Vanhee et al. Cell Immunol. 155: 446-456 (1994)). The observation that oat extracts dose-dependently reduced production of these cytokines in the IL-1β stimulated HAEC is novel and provides yet another potential mechanism by which oat may reduce the risk of atherosclerosis in vivo.

### Example 6

### Effect of Avenanthramide on FBS-Induced Cell Proliferation of HSMC

[³H] thymidine incorporation into DNA was used to determine the effect of Avenanthramide on cell proliferation. Synthetic Av-C was used in this experiment. Human aortic smooth muscle cells (HSMC) were seeded into 24-well plate at equal density. After reaching to 80% confluency, the cells were synchronized to quiescent condition by serum starvation for 48 h. Cells were then stimulated with 10% of FBS in the absence or presence of different concentration of Av for 24 h. During the last 4 h of incubation, 1.0 μCi/mL of [³H] thymidine was added to each well. DNA was precipitated with 10% trichloroacetic acid and solubilized with 0.1N sodium hydroxide and counted in a scinitillation counter. Treatment of HSMC with Avenanthramide inhibited serum-induced DNA synthesis and thus cell proliferation (FIG. 7). The inhibitory effect of Avenanthramide was concentration-dependent. At concentration of 120 μM, Avenanthramide inhibited more than 50% of cell proliferation without significant cytotoxity as determined by trypan blue cell viability assay.

The cell numbers were counted to verify that the results observed with the [³H] thymidine incorporation were reflective of changes in cell growth. HSMC or A10 cells (rat embryonic aortic smooth muscle cell) were seeded into 6-well plate at a density of 0.5×10⁴/well. Four hr later 120 μM of avenanthramide were added to each well. At different time points (2d, 3d and 4d) the cells were trypsinzied and the total cell number were counted using a hemocytometer. Trypan blue exclusion test was carried out to determine the cell viability.

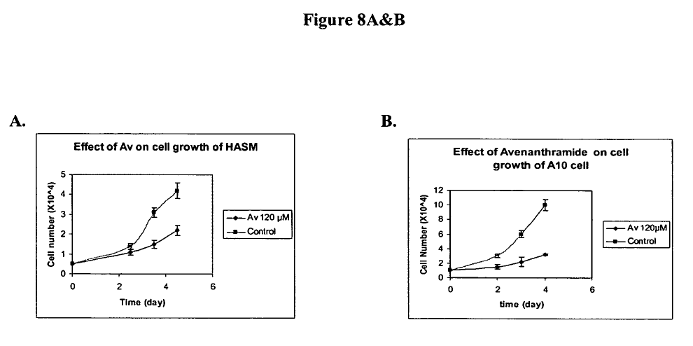

As shown in FIG. 8, 10% FCS caused a rapid increase of cell number, the doubling time for HSMC and A10 are calculated to be 28 hr and 38 hr, respectively. The addition of 120 μM Avenanthramide attenuated this cell number increase and their doubling time increased to more than 48 hr. This result is consistent with the result obtained from [³H] thymidine incorporation. Avenanthramide seemed to have more inhibitory effect for A10 cell than for HSMC. When Avenanthramide was removed from cell culture medium, the cells proliferation restarted again (data not shown), indicating that the inhibitory effect was reversible.

### Example 7

### Effect of Avenanthramides on NO Production

In addition to its vasodilating feature, NO has antiatherosclerotic properties, such as prevention of platelet aggregation, leukocyte adhesion, smooth muscle cell proliferation, and the expression of genes involved in atherogenesis. In this study, the human aortic smooth muscle cells (HSMC) and human aortic endothelia cells (HEC) were used to determine if avenanthramides can influence the NO production level. Synthetic Av-C was used in this experiment. The quantification of NO production was performed by the 4,5 diaminofluorescein (DAF-2) fluorescence assay. Briefly, cells were seeded into 24-well plate and let them grow to 90% confluent condition. The cells were pretreated with different concentrations of Avenanthramide for 24 h. The culture medium was then changed to PBS containing 100 μL-arginine, 1 μM bradykinin and L-NAME. Then 0.1 μM of DAF-2 was added to each well. After another 2 h of incubation, 200 μL of supernatants were transferred to black microplates and the fluorescence was measured with a fluorescence microplate reader calibrated for excitation at 485 nm and emission at 538 nm.

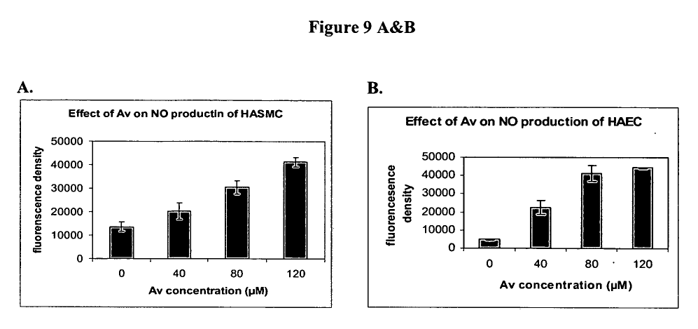

As shown in FIG. 9, Avenanthramide dose-dependently and significantly induced NO production of both HSMC and HAEC. For these two cells, at concentration of 120 μM, Avenanthramide respectively induced 3.0 folds and 8.8 folds increase in NO production compared with control (P<0.05).

### Example 8

### Effect of Avenanthramide on eNOS mRNA Level

Since NO is catalyzed by endothelium nitric oxide synthases (eNOS), we next examined the role of Avenanthramide in induction of eNOS expression level by real time PCR. Total mRNA was extracted using RNesy Mini Kit (QIAGEN) and quantified spectrophotometrically at 260 nm. Twenty μl of first-strand cDNA was synthesized from 1.5 μg of total RNA by using 300 ng of random hexanucleotide primers and 200 u of SuperScript II (Invitrogen) at 42 C for 1 h followed by heat inactivation of reverse transcriptase at 70 C for 15 min. Fifty μL of PCR reaction mixture contained 5 μL cDNA, up- and down-stream primers (200 nM) and 25 μL of SYBR Green was set up into the ABI 4400 to perform the amplification for 40 cycles with denaturation at 95 C for 30 sec, annealing at 60 C for 1 min and extension at 72 C for 1 min. Primers designed based on the published gene sequences were synthesized from Tufts University Core Facility. The primer sequences used for eNOS were:

5′-ATCCTGGCAAGCCCTAAGACC-3′(upstream)
and
5′-TGGTAGCGTTTGCTGATCCCG-3′(downstream).

Beta-actin was used as an internal control with the primers:

5′-TTGTAACCAACTGGGACGATATGG-3′(upstream),
5′-CACAATGCCAGTGGTACGACC-3′(downstream).

Beta-actin mRNA expression level as an internal control. As shown in FIG. 4, compare with control, treatment with Avenanthramide increased the eNOS mRNA expression level. The enhancing effect is dose-dependent and the pattern is consistent with the results from NO production (FIG. 3).

TABLE 1
Effect of Avenanthramide on eNOS mRNA expression level

| Av. Con. | eNos Average | | ΔCt | Fold |
|---|---|---|---|---|
| (μM) | CT | β-actin CT | eNOS-β-actin | Change |
| 0 | 35.6 ± 0.21 | 27.9 ± 0.25 | 7.67 ± 0.33 | 1 |
| | | | | (0.67-1.33) |
| 40 | | 28.8 ± 0.34 | 7.0 ± 0.51 | 1.56 |
| | | | | (1.22-1.9) |
| 80 | | 29 ± 0.33 | 6.41 ± 0.48 | 2.39 |
| | | | | (2.1-2.7) |

1. A method for modulating nitric oxide (NO) levels, comprising:
administering an effective amount of a substantially purified phenolic composition comprising at least one member selected from the group consisting of compounds of formula: 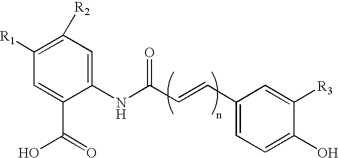
wherein n is less than or equal to six and R₁, R₂, and R₃ are the same or different and selected from the group comprising a hydrogen, a hydroxide, an aliphatic group, an aromatic group, an acyl group, an alkoxy group, an alkylene group, an alkenylene group, an alkynylene group, a hydroxycarbonylalkyl group, an anhydride, an amide, an amine, and a heterocyclic aromatic group,
whereby administration modulates nitric oxide production in vascular cells.

2. The method of claim 1, wherein n is less than three and R₁, R₂, and R₃ are selected from the group consisting of H, OH, and OCH₃.

3. The method of claim 1, wherein the method further comprises administering at least one compound that is an extract or concentrate of oat grain.

4. The method of claim 1, wherein the method further comprises administering at least one compound that is produced synthetically.

5. A pharmaceutical composition comprising a compound of claim 1, or a pharmaceutically acceptable salt thereof, and a pharmaceutically acceptable carrier or diluent.

6. The method of claim 1, wherein the method further comprises modulating at least one of endothelial nitric oxide synthase (eNOS), neuronal nitric oxide synthase (NNOS), and inducible NOS (iNOS).

7. The method of claim 1, wherein the method further comprises increasing endothelial nitric oxide synthase (eNOS).

8. The method of claim 1, wherein the administration of the phenolic composition modulates nitric oxide production in at least one of human aortic smooth muscle cells and vascular endothelial cells.

9. The method of claim 1, wherein the administration of the substantially purified phenolic composition increases nitric oxide production.

10. The method of claim 1, wherein the method further comprises modulating a condition associated with a lack of or need for nitric oxide (NO) by increasing NO concentration in a subject.

11. A method for inhibiting smooth muscle cell proliferation in a subject comprising
administering to a subject an effective amount of a substantially purified composition of at least one member selected from the group consisting of compounds of formula: 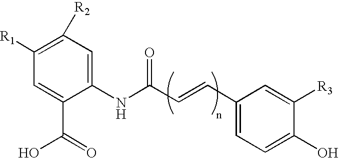
wherein n is less than or equal to six and R₁, R₂, and R₃ are the same or different and selected from the group comprising a hydrogen, a hydroxide, an aliphatic group, an aromatic group, an acyl group, an alkoxy group, an alkylene group, an alkenylene group, an alkynylene group, a hydroxycarbonylalkyl group, an anhydride, an amide, an amine, and a heterocyclic aromatic group.

12. The method of claim 11, wherein n is less than 3 and R₁, R₂, and R₃ can be selected from the group consisting of H, OH, or OCH₃.

13. A method for modulating an immune response in a subject, comprising:
administering to a subject an effective amount of a substantially purified phenolic composition of formula, 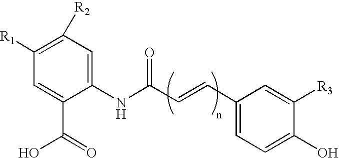
wherein n is less than or equal to six and R₁, R₂, and R₃ are the same or different and selected from the group comprising a hydrogen, a hydroxide, an aliphatic group, an aromatic group, an acyl group, an alkoxy group, an alkylene group, an alkenylene group, an alkynylene group, a hydroxycarbonylalkyl group, an anhydride, an amide, an amine, and a heterocyclic aromatic group,
whereby administration modulates at least one pro-inflammatory molecule or cell adhesion molecule.

14. The method of claim 13, wherein n is less than three and R₁, R₂, and R₃ are selected from the group consisting of H, OH, and OCH₃.

15. The method of claim 13, wherein the method further comprises administering at least one compound that is an extract or concentrate of oat grain.

16. The method of claim 13, wherein the method further comprises administering at least one compound that is produced synthetically.

17. The method of claim 13, wherein the at least one cell adhesion molecule is selected from the group consisting of ICAM-1, VCAM-1, and E-selectin.

18. The method of claim 13, wherein the at least one pro-inflammatory molecule is selected from the group consisting of IL-6, IL-8, and MCP-1.

19. The method of claim 13, wherein the method further comprises decreasing at least of pro-inflammatory molecule or cell adhesion molecule.

20. A therapeutic composition for use in reducing an immune response in a subject when administered in an effective amount to modulate at least one of the group consisting of pro-inflammatory molecules, cell adhesion molecules, nitric oxide levels, and smooth muscle cell proliferation,
wherein the therapeutic composition comprises a substantially purified phenolic composition with at least one member selected from the group consisting of compounds of formula: 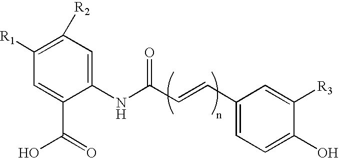
wherein n is less than or equal to six and R₁, R₂, and R₃ are the same or different and selected from the group comprising a hydrogen, a hydroxide, an aliphatic group, an aromatic group, an acyl group, an alkoxy group, an alkylene group, an alkenylene group, an alkynylene group, a hydroxycarbonylalkyl group, an anhydride, an amide, an amine, and a heterocyclic aromatic group.

21. The therapeutic composition of claim 20, wherein n is less than three and R₁, R₂, and R₃ are selected from the group consisting of H, OH, and OCH₃.
